(19) United States
(12) Patent Application Publication
(10) Pub. No.: US 2023/0321163 A1
(43) Pub. Date: Oct. 12, 2023

(54) Composition For Treating or Preventing Clostridium Difficile Infection

(71) Applicant: Seoul National University R&DB Foundation, Seoul (KR)

(72) Inventors: Gwangpyo Ko, Seoul (KR); Hyun Ju You, Incheon (KR); Jun Sun Yu, Seoul (KR); Su Eun Choe, Seoul (KR)

(21) Appl. No.: 18/042,378

(22) PCT Filed: Aug. 20, 2021
(86) PCT No.: PCT/KR2021/011141

§ 371 Date: Feb. 21, 2023

(30) Foreign Application Priority Data
Aug. 21, 2020 (KR) 1020200105430

(51) Int. Cl. A61K 35/744; A61K 35/742; A61K 35/741; A61P 31/04

(52) U.S. Cl. CPC A61K 35/744; A61K 35/742; A61K 35/741; A61P 31/04

(57) ABSTRACT

The present invention relates to a composition for preventing or treating Clostridium difficile infection, comprising at least one from the group consisting of cells, cultures, lysates, and extracts of Clostridium scindens, Blautia producta, and Enterococcus faecium.

### TECHNICAL FIELD

The present application relates to a composition for treating or preventing Clostridium difficile infection, including Clostridium scindens, Blautia producta and Enterococcus faecium, and further including one or more of Blautia faecis and Proteus terrae.

### BACKGROUND ART

Clostridium difficile is an anaerobic spore-forming gram-positive pathogen, which is one of clinically important bacteria of the genus Clostridium that forms colonies in the gastrointestinal tract and then produces toxins that cause diarrhea and, in severe cases, cause pseudomembranous colitis.

Pathogenic Clostridium difficile produces toxins known as enterotoxin (toxin A), cytotoxin (toxin B), and binary toxin, resulting in severe diarrhea, toxic megacolon, perforation, sepsis, and pseudomembranous colitis. In particular, toxin A causes secretory, hemorrhagic diarrhea, and toxin B, as cytotoxin, exhibits destructive cytopathic effects in tissue culture cells.

Various antibiotics are known to be associated with Clostridium difficile-associated disease (CDAD). Intestinal acquisition of Clostridium difficile has been found to occur in approximately 10-25% of hospitalized patients and increases with length of hospitalization. The pathogenic Clostridium difficile may survive for a long time outside the body by forming spores after cells are excreted from the diarrhea of infected patients. Therefore, the Clostridium difficile may survive in environment for a long period of time, and in turn, spores may cause additional infections through the mouth, resulting in outbreaks of Clostridium difficile in hospitals.

Currently, standard therapy used for treatment of Clostridium difficile infection (CDI) is antibiotic prescription, but it is known to have a relatively low treatment success rate and high recurrence rate. Metronidazole and vancomycin have a limited treatment success rate even during the first treatment, with a recurrence rate of 20-30%, a treatment success rate of 70% for the first relapse, and a decrease to 35% for more recurrences, and the drugs is not suitable for use in refractory severe CDI and recurrent CDI caused by high toxic strains. In addition, fidaxomicin inhibits C. difficile toxin A, B synthesis and spore formation, and a recurrence rate is also lower than other antibiotics, but the price is high, and nausea, vomiting, fever, dizziness, and increased liver enzyme levels are still matters of interest. The possibility of nitazoxanide as a therapeutic agent has also been suggested, but related studies are still lacking.

Recently, non-antibiotic therapy using fecal microbiota transplantation (FMT), vaccines, and administration of core microbes has been proposed for prevention and treatment of CDI. FMT, in which stool from a healthy donor is administered to the patient's intestinal tract, is one of therapies for refractory or recurrent CDI, and as a result of 28 studies on 317 CDI patients, it was reported that it showed a 92% recovery rate and high recurrence prevention effect. However, despite the high treatment success rate and low recurrence rate, the FMT therapy has several limitations, such as a procedure process that may be unpleasant for the general public to endure, non-standardized treatment, and pathogen propagation. C. difficile vaccines, including toxoids A and B, are also being studied to show efficacy in patients with recurrent CDI, but commercialization is expected to take considerable time.

On the other hand, beneficial microorganisms isolated from intestine may cause changes in the intestinal ecosystem, and as a result, maintain spatial and resource competition with C. difficile. It has been reported that when treating them, they actually show the same level of therapeutic effect as metronidazole or vancomycin, and the recurrence rate is also significantly lowered (Ollech et al., Best Practice & Research Clinical Gastroenterology, 2016, Vol. 30, No. 1, pp. 111-118). Therefore, the process of securing and mass-producing functional beneficial strains that may make a great contribution to the prevention and treatment of CDI has a very important significance as a method that may supplement/replace antibiotic therapy.

Currently, there are known examples of attempts to treat CDI using a certain strain combination (for example, Korean Patent Publication No. 2019-0030687), or attempts to prevent or treat bacterial infection along with autoimmune diseases, inflammatory diseases, and the like by inducing proliferation and/or accumulation of regulatory T cells to suppress excessive inflammation caused by immunity (European Patent No. 2 575 835). In addition, Ceres Therapeutics and Vedanta Biosciences are conducting clinical trials on CDI therapeutic agent using probiotics, but CDI therapeutic agents including probiotics as an active ingredient have not yet been commercialized.

Accordingly, the present inventors have made research efforts to discover bacteria capable of preventing or treating Clostridium difficile infection, and as a result, Clostridium scindens KBL987 (KCTC13277BP) having a Clostridium difficile growth inhibitory effect was discovered (Korean Patent Application No. 2017-0122608).

Furthermore, the present inventors continued research to find strains exhibiting a CDI inhibitory effect in combination with Clostridium scindens, and as a result, completed the present invention by finding that an excellent CDI inhibitory effect may be achieved when Blautia producta and Enterococcus faecium, and additional one or more of Blautia faecis and Proteus terrae are used in combination with Clostridium scindens.

### RELATED ART DOCUMENT

### [Patent Document]

(Patent Document 1) Korean Patent Publication No. 2019-0030687
(Patent Document 2) European Patent No. 2 575 835

### [Non-Patent Document]

(Non-Patent Document 1) Ollech et al., Best Practice & Research Clinical Gastroenterology, 2016, Vol. 30, No. 1, pp. 111-118

### DISCLOSURE OF INVENTION

### Technical Goals

The present disclosure is for the purpose of providing a pharmaceutical composition for preventing or treating Clostridium difficile infection, including one or more selected from the group consisting of cells, cultures, lysates, and extracts of Clostridium scindens, Blautia producta, and Enterococcus faecium.

The present disclosure is also for the purpose of providing a food composition for preventing or improving Clostridium difficile infection, including one or more selected from the group consisting of cells, cultures, lysates, and extracts of Clostridium scindens, Blautia producta, and Enterococcus faecium.

### Technical Solution

The present inventors have made research efforts to discover a strain combination having excellent preventive or therapeutic effects on Clostridium difficile infection. As a result, the present invention was completed by experimentally proving that combination of Clostridium scindens, Blautia producta and Enterococcus faecium, and optionally one or more Blautia faecis and Proteus terrae exhibits excellent preventive and therapeutic effects against Clostridium difficile infection.

As one embodiment of the present disclosure, the present disclosure relates to a pharmaceutical composition for preventing or treating Clostridium difficile infection, including one or more selected from the group consisting of cells, cultures, lysates, and extracts of Clostridium scindens, Blautia producta, and Enterococcus faecium.

As another aspect of the present disclosure, the present disclosure relates to a food composition for preventing or improving Clostridium difficile infection, including one or more selected from the group consisting of cells, cultures, lysates, and extracts of Clostridium scindens, Blautia producta, and Enterococcus faecium.

As another example of the present disclosure, the pharmaceutical composition or food composition of the present disclosure may further include one or more selected from the group consisting of cells, cultures, lysates, and extracts of one or more of Blautia faecis and Proteus terrae.

Hereinafter, the present disclosure will be described in more detail.

Unless defined otherwise, all technical and scientific terms used herein have the same meaning as commonly understood by one of ordinary skill in the art to which the present disclosure belongs. In general, the nomenclature used herein is those well known and commonly used in the art.

A pharmaceutical composition for preventing or treating Clostridium difficile infection and a food composition for preventing or improving Clostridium difficile infection according to the present disclosure include one or more selected from the group consisting of cells, cultures, lysates, and extracts of Clostridium scindens, Blautia producta, and Enterococcus faecium.

In the present disclosure, the term “cell” includes both live cells and dead cells sterilized by heating, pressurization, or drug treatment. Preferably, the composition of the present disclosure includes purified cells.

In the present disclosure, the term “culture” refers to a product obtained by culturing a strain in a known medium, and the product may include the strain. The medium may be selected from known liquid medium or solid medium, and may be, but not limited to, for example, Cholate agar medium, yBHI agar medium, GAM agar medium, MRS agar medium, MRS liquid medium, GAM liquid medium, and the like. An appropriate medium may be selected according to the strain, for example, Cholate agar medium for Proteus terrae and Enterococcus faecium, yBHI agar medium for Blautia producta and Blautia faecis, and GAM agar medium for Clostridium scindens may be selected.

In the present disclosure, the term “lysate” means that cells are destroyed by enzyme treatment, homogenization, or ultrasonic treatment, the term “extract” refers to a product obtained by extracting a strain with any appropriate extraction solvent, and a suitable extraction solvent may be selected according to a type of strain.

In the present disclosure, Clostridium scindens may preferably have a 16s rDNA sequence that is 97% or more, 98% or more, 99% or more, or 100% identical to SEQ ID NO: 1.

In addition, Clostridium scindens of the present disclosure may be Clostridium scindens KBL987 strain with deposit number KCTC13277BP. The strain was deposited with the Korean Collection for Type Culture on May 29, 2017 as “Clostridium scindens SNUG 40402,” but was renamed “Clostridium scindens KBL987” on Jul. 26, 2019.

In the present disclosure, Blautia producta may preferably have a 16s rDNA sequence that is 97% or more, 98% or more, 99% or more, or 100% identical to any one of SEQ ID NOs: 2 to 5.

In addition, the Blautia producta of the present disclosure may preferably be Blautia producta ATCC27340 strain with deposit number KCTC15607, Blautia producta KBL988 strain with deposit number KCTC13915BP, Blautia producta KBL990 strain with deposit number KCTC13917BP or Blautia producta KBL991 strain with deposit number KCTC13918BP, deposited with Korean Collection for Type Culture in 2008.

In the present disclosure, Enterococcus faecium may preferably have a 16s rDNA sequence that is 97% or more, 98% or more, 99% or more, or 100% identical to SEQ ID NO: 6.

In addition, the Enterococcus faecium of the present disclosure may preferably be Enterococcus faecium KBL986 strain with deposit number KCTC13914BP.

On the other hand, a pharmaceutical composition for preventing or treating Clostridium difficile infection and a food composition for preventing or improving Clostridium difficile infection according to the present disclosure may further include Clostridium scindens, Blautia producta, and Enterococcus faecium, and optionally one or more of Blautia faecis and Proteus terrae. Preferably, the composition according to the present disclosure may include both Blautia faecis and Proteus terrae.

In the present disclosure, Blautia faecis may preferably have a 16s rDNA sequence that is 97% or more, 98% or more, 99% or more, or 100% identical to SEQ ID NO: 7.

In addition, the Blautia faecis of the present disclosure may preferably be Blautia faecis KBL989 strain with deposit number KCTC13916BP.

In the present disclosure, Proteus terrae may preferably have a 16s rDNA sequence that is 97% or more, 98% or more, 99% or more, or 100% identical to SEQ ID NO: 8.

In addition, the Proteus terrae of the present disclosure may preferably be Proteus terrae KBL985 strain with deposit number KCTC13933BP.

The pharmaceutical composition and food composition according to the present disclosure exhibit excellent effects in inhibiting body weight loss and improving survival rate in case of infection with Clostridium difficile, and since the composition significantly reduces amounts of stool c.f.u. and stool toxin, it has excellent efficacy in preventing, treating or improving Clostridium difficile.

In the present disclosure, the term “Clostridium difficile infection” or “CDI” encompasses Clostridium difficile infection or antimicrobial-associated diarrhea expressed in association therewith, an intestinal disease caused by Clostridium difficile, an inflammation of a gastrointestinal tract, and the like. A variety of factors, including use of antibiotics, may lead to intestinal imbalances in the gastrointestinal tract, which may allow colony formation by pathogenic microorganisms such as C. difficile. Such colony formation or pathogenic infection may result in various side effects in the subject, including diarrhea, which is one of the characteristic main symptoms of CDI. In the case of CDI, diarrhea is believed to be a result of Toxin B production of C. difficile, which opens tight junctions between intestinal epithelial cells, increasing vascular permeability, bleeding and inflammation.

In the present disclosure, symptoms of Clostridium difficile infection may range from mild to severe and include diarrhea, fever and painful abdominal cramping. Clostridium difficile infection may also lead to life-threatening complications, such as extreme swelling of intestine due to accumulation of gas (toxic megacolon). Clostridium difficile-associated disease (CDAD) includes widespread diarrheal diseases caused by toxins produced from Clostridium difficile, including severe colitis regardless of presence of a pseudomembrane.

The pharmaceutical composition and food composition of the present disclosure may be administered to treat infection in a subject suffering from Clostridium difficile infection or in a subject who has received treatment for Clostridium difficile infection but has relapsed infection. A subject suffering from Clostridium difficile infection may be an asymptomatic carrier. In addition, the composition of the present disclosure may be administered for prophylactic purposes to subjects at risk of infection with Clostridium difficile, such as subjects who have had a pathogenic infection, subjects who have been treated with antibiotics, and subjects undergoing procedures that increase the risk of acquiring a pathogenic infection (for example, surgery and/or hospitalization).

As used herein, the term “preventing” refers to averting, delaying, impeding, or hindering Clostridium difficile infection and related diseases, symptoms, and the like by administration of the pharmaceutical composition of the present disclosure.

In the present disclosure, the term “treating” refers to improving, curing, reducing Clostridium difficile infection and related diseases, symptoms, and the like, or reducing or stopping the progression of the diseases by administration of the pharmaceutical composition of the present disclosure.

The pharmaceutical composition and food composition of the present disclosure may include a strain combination of the present disclosure in any form, for example in aqueous form, such as a solution or suspension embedded in a semi-solid form, in powder form or in lyophilized form. In some embodiments, the composition or some or all strains of the composition are lyophilized. Methods for lyophilizing compositions, particularly compositions including strains, are well known in the art (see, for example, U.S. Pat. Nos. 3,261,761; 4,205,132; PCT Publications WO 2014/029578 and WO 2012/098358). A strain combination of the present disclosure may be lyophilized as a combination and/or lyophilized separately and combined before administration. In addition, each strain may be combined with a pharmaceutically acceptable excipient before combining with another strain, or multiple lyophilized bacteria may be combined while remaining in lyophilized form, and once combined, a mixture of bacteria may be subsequently combined with pharmaceutical excipients. In some embodiments, some or all of the strains may be a lyophilized cake.

Each strain of the present disclosure may be produced using fermentation techniques well known in the art. In some embodiments, an active ingredient is manufactured using an anaerobic fermenter capable of supporting rapid growth of anaerobic bacterial species. An anaerobic fermenter may be, for example, a stirred tank reactor or a disposable wave bioreactor. Fermentation products may be purified and concentrated by techniques known in the art, such as centrifugation and filtration, and optionally dried and lyophilized by techniques well known in the art.

When the composition of the present disclosure is prepared as a pharmaceutical composition, the pharmaceutical composition of the present disclosure includes a pharmaceutically acceptable carrier. Pharmaceutically acceptable carriers included in the pharmaceutical composition of the present disclosure are commonly used in formulation, and includes, but not limited to, lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methylhydroxybenzoate, propylhydroxybenzoate, talc, magnesium stearate and mineral oil. The pharmaceutical composition of the present disclosure may further include, in addition to the above components, lubricants, wetting agents, sweetening agents, flavoring agents, emulsifying agents, suspending agents, preservatives, and the like. Suitable pharmaceutically acceptable carriers and agents are described in detail in Remington's Pharmaceutical Sciences (19th ed., 1995).

The pharmaceutical composition of the present disclosure may be administered orally or parenterally. For example, the pharmaceutical composition of the present disclosure may be preferably administered orally, intrarectally or intravenously.

A suitable dosage of the pharmaceutical composition of the present disclosure may be variously prescribed according to factors such as formulation method, administration mode, patient's age, body weight, sex, pathological condition, food, administration time, administration route, excretion rate, and response sensitivity. The pharmaceutical composition of the present disclosure may be administered to a subject one or more times a day, such as twice a day, three times a day, and the like. Unit dose means physically separated units suitable for unit dosage for a subject, each unit includes a pharmaceutical carrier, and a predetermined amount of a strain combination of the present disclosure showing therapeutic effects.

A general dosage of the pharmaceutical composition of the present disclosure is within the range of 0.001 to 10 g, preferably 0.01 to 5 g per dose. When administered in this way, the strain combination of the present disclosure may be administered at 1×10³ cfu/day to 1×10¹¹ cfu/day, preferably 1×10⁷ cfu/day to 1×10¹⁰ cfu/day, more preferably 1×10⁹ cfu/day.

The pharmaceutical composition of the present disclosure may be prepared in a unit dosage form by formulating using a pharmaceutically acceptable carrier and/or excipient, or may be prepared by incorporation into a multi-dose includeer, according to a method that may be easily carried out by a person of ordinary skill in the art to which the present disclosure pertains. The form of the preparation may be in the form of solutions in or aqueous medium, suspensions, syrups, or emulsions, or in the form of an extract, powder, granule, tablet or capsule, and may additionally include a dispersant or stabilizer.

Preferably, the pharmaceutical composition of the present disclosure is formulated for delivery to intestine (for example, small intestine and/or colon). In some embodiments, bacteria are formulated with an enteric coating that increases survival of bacteria in the harsh environment of stomach. An enteric coating is one that resists the action of gastric juice in the stomach, allowing bacteria that become incorporated therein to pass through the stomach and into the intestine. The enteric coating may be made of polymers and copolymers well known in the art, such as commercially available EUDRAGIT (Evonik Industries).

In another aspect, the present disclosure relates to a food composition for preventing or improving Clostridium difficile infection, including one or more selected from the group consisting of cells, cultures, lysates, and extracts of Clostridium scindens, Blautia producta, and Enterococcus faecium, and optionally one or more selected from the group consisting of cells, cultures, lysates, and extracts of one or more of Blautia faecis and Proteus terrae.

The food composition of the present disclosure may be easily utilized as a food that is effective in preventing or improving CDI, and the food includes, but not limited to, a main raw material or a supplementary raw material of food, a food additive, a health functional food or a functional beverage.

The food composition means a natural product or processed product including one or more nutrients, and preferably means a state that may be eaten directly through a certain amount of processing process.

When the composition of the present disclosure is provided in the form of a food composition, the composition of the present disclosure may include, in addition to the active ingredient, ingredients commonly added during food production, for example, protein, carbohydrate, fat, nutrients, seasoning and flavoring agents. Examples of the above-mentioned carbohydrates include monosaccharides in the related art such as glucose, fructose, and the like, disaccharides such as maltose, sucrose, and the like, oligosaccharides and polysaccharides, for example, sugars in the related art such as dextrins and cyclodextrins, and sugar alcohols such as xylitol, sorbitol and erythritol. As the sweetener, natural sweeteners (thaumatin, Stevia extract, rebaudioside A, glycyrrhizin, and the like) and synthetic sweeteners (saccharin, aspartame, and the like) may be used. For example, when the food composition of the present disclosure is prepared as a drink, citric acid, high fructose corn syrup, sugar, glucose, acetic acid, malic acid, fruit juice, Eucommia extract, jujube extract, licorice extract, or the like may be further included in addition to the active ingredient of the present disclosure.

The food composition according to the present disclosure may be produced using a method known in the art, and may include the same amount of cells (for example, based on weight, amount or CFU) as the pharmaceutical composition of the present disclosure. For example, the food composition according to the present disclosure may include a strain combination of the present disclosure in an amount of 0.001% to 100% by weight, preferably 1% to 99% by weight of the total food weight, and in the case of a beverage, may be included in a ratio of 0.001 g to 10 g per 100 mL, preferably 0.01 g to 1 g per 100 mL. The amount of cells in food may depend on a variety of factors, including the capacity of the food, frequency of consumption of the food, the type of strain included in the food, the amount of moisture in the food, and/or additional conditions for the survival of the strain in the food.

In preparing the food composition of the present disclosure, together with the strain combination of the present disclosure, any probiotic strain which is suitable for human or animal intake, and capable of inhibiting pathogenic harmful bacteria or improving the microbial balance in the mammalian intestinal tract upon intake may be used. Examples of such probiotic microorganisms include, but not limited to, bacteria of the genuses Lactobacillus, Bifidobacterium, Leuconostoc, Lactococcus, Bacillus, and Streptococcus.

The pharmaceutical composition and/or food composition of the present disclosure may further include a cryoprotectant. The cryoprotectant may be a non-naturally occurring material or a naturally occurring material, and during freeze-drying of the composition or microbial strains included therein, when the cryoprotectant is a material which prevents or reduces destruction, damage of the microorganisms, or maintains the original activity by preventing or reducing the decrease and loss of activity of microorganisms caused by freeze-drying, it can be applied without limitation to its type. For example, the cryoprotectant may include, but not limited to, trehalose, glycerol, maltodextrin, skim milk, starch, soy flour, saccharide, amino acid, peptide, gelatin, glycerol, sugar alcohol, whey, alginic acid, ascorbic acid, yeast extract, garlic extract, and the like. The cryoprotectant may be included, but not limited to, in an amount of 0.01% to 20% by weight or 0.01% to 10% by weight, based on the total weight of the composition. The pharmaceutical composition and/or food composition of the present disclosure may be provided as a preparation in a lyophilized form as it further includes the cryoprotectant, and thus can show more advantageous advantages and effects for storage, preservation, transport, movement, distribution or intake of the composition.

Another aspect of the present disclosure provides a method for preventing or treating Clostridium difficile, including administering a pharmaceutically effective amount of the strain combination of the present disclosure to a subject in need of prevention or treatment of Clostridium difficile.

The subject in need of prevention or treatment of the diseases includes all animals including humans. For example, it may be an animal such as a dog, a cat, or a mouse, preferably a human.

Another aspect of the present disclosure provides use of the strain combination or the composition of the present disclosure for use in prevention or treatment of Clostridium difficile, and use of the strain combination or the composition for the preparation of a prophylactic or therapeutic agent for Clostridium difficile.

Since the pharmaceutical composition and administration method used in the method for preventing or treating the disease have been described above, descriptions of common contents between the two are omitted in order to avoid excessive complexity of the present specification.

The present disclosure is not limited in its application to the details of the arrangement and construction of components set forth in the following description or illustrated in the drawings. The present disclosure is capable of other embodiments and of being practiced or carried out in various ways. In addition, the phraseology and terminology used herein is for the purpose of description and should not be regarded as limiting. The use of “comprise,” “comprising” or “having,” “containing,” “comprised” and variations thereof herein is meant to encompass the items listed thereafter and equivalents thereof, as well as additional items.

Unless defined otherwise herein, scientific and technical terms used in connection with the present disclosure should have the meanings commonly understood by one of ordinary skill in the art. The methods and techniques of the present disclosure are generally performed according to methods in the related art which are well known in the related art. In general, the nomenclature and techniques used in connection with biochemistry, enzymology, molecular and cell biology, microbiology, virology, cell or tissue culture, genetics, and protein and nuclear chemistry described herein are well known in the art and commonly will be used The methods and techniques of the present disclosure are generally performed in accordance with conventional methods well known in the art, and performed as described in various general and more specific references cited and discussed throughout this specification, unless otherwise indicated.

### Advantageous Effects

A strain combination of the present disclosure including Clostridium scindens, Blautia producta, and Enterococcus faecium, and optionally one or more of Blautia faecis and Proteus terrae, exhibits excellent body weight loss inhibition and survival rate improvement effects, and significantly reduces an amount of stool c.f.u. and stool toxin when infected with Clostridium difficile, and thus can be usefully used for prevention and treatment of Clostridium difficile infection.

### DESCRIPTION OF DRAWINGS

FIG. 1 is a schematic diagram of an animal model for testing the inhibitory effect of C. difficile infection of a strain combination of the present disclosure.

FIG. 2 shows body weight loss rate (FIG. 2A and FIG. 2B, respectively) and survival rate (FIG. 2C) 48 hours and 72 hours after infection with C. difficile spores in the animal model of FIG. 1.

FIG. 3 shows a body weight change rate and survival rate over time when each strain is administered single and in combination with Cs in the animal model of FIG. 1.

FIG. 4 is a schematic diagram of another animal model for testing the inhibitory effect of C. difficile infection of a strain combination of the present disclosure.

FIG. 5 shows body weight change over time after infection with C. difficile spores in the animal model of FIG. 4.

FIG. 6 shows a result of measuring body weight change (FIG. 6A and FIG. 6B, respectively) and survival rate (FIG. 6C) 48 hours and 72 hours after infection with spores in the animal model of FIG. 4.

FIG. 7 shows a result of a comparative experiment on body weight loss rate and survival rate of a combination of five strains and a combination of four strains according to the present disclosure.

FIG. 8 shows a result of measuring body weight loss inhibitory effect according to dosage change of a strain combination according to the present disclosure.

FIG. 9 shows a result of measuring body weight change over time in a strain combination of the present disclosure using various Blautia producta strains (FIG. 9A), body weight change after 48 hours and 72 hours after spore infection (FIGS. 9B and 9C, respectively), survival rate (FIG. 9D), stool c.f.u. after 24 hours. and stool toxin (FIG. 9E and FIG. 9F, respectively).

FIG. 10 shows a result of measuring and comparing a CDI infection inhibitory effect of an optimal strain combination (CBBE: Cs+Bp+Bf+Ef) of the present disclosure and other strain combination by various methods. FIG. 10A shows results of measuring body weight changes, survival rate, stool c.f.u. after 24 hours, and stool toxin over time when the optimal strain combination and Cs strain single were administered. FIG. 10B shows a result of comparing body weight changes when different strain combinations are administered, and FIGS. 10C to 10E shows results of measuring the number of strains of C. difficile, stool c.f.u. and stool toxin.

FIG. 11 shows experimental results confirming the effect of an optimal strain combination (CBBE: Cs+Bp+Bf+Ef) of the present disclosure through ex vivo experiments, and the results was compared with Bp+Bf+Ef (ΔCS), Cs+Bf+Ef (ΔBP), Cs+Bp+Ef (ΔBF), and Cs+Bp+Bf (ΔEF) combinations or Cs single inoculated results. Relative amount of C. difficile (FIG. 11A), amount of a secondary bile acid (DCA) (FIG. 11B), pH measurement result (FIG. 11C), correlation between each strain in CBBE and pH (FIG. 11D), and negative correlation between each strain in CBBE and C. difficile (FIG. 11E) are shown.

FIGS. 12 to 15 show experimental schematics and experimental results for confirming an effect of a combination of four strains according to the present disclosure on Treg cells.

### MODES OF THE INVENTION

The present disclosure is further illustrated by the following examples, which should not be construed as further limiting in any way. The entire contents of all references (including references, issued patents, published patent applications, and pending patent applications) cited throughout this application are expressly incorporated herein by reference, particularly for the teachings mentioned above. However, citation of any reference is not an admission that the reference is the related art.

### Example 1. Candidate Strain and Clostridium difficile Culture

1-1. Culture of Candidate Strain

In order to identify strains that are effective in treating or improving symptoms of Clostridium difficile infection (CDI), Proteus terrae and Enterococcus facium were cultured on Cholate agar, Blautia producta and Blautia faecis were cultured on yBHI agar, and Clostridium scindens was cultured on GAM agar. It was used in the experiment after activation through subculture a total of 2 times at 24 hour intervals. Deposit information of the strains used in the examples is as follows. As shown in Table 1 below, Clostridium scindens is sometimes written as ‘Cs’, Blautia producta as ‘BpKCTC or Bp, BpYA44, BpMG11, or BpMA68’ according to the type of each strain, respectively, and Enterococcus faecium as ‘Ef’, Blautia faecis as ‘Bf’, and Proteus terrae as ‘Pt’.

TABLE 1

| | | Deposit number | Date of deposit | Abbreviation | |
|---|---|---|---|---|---|
| Clostridium scindens KBL987 | | KCTC 13277BP | 2017 May 29 | Cs | |
| Blautia | ATCC27340 | KCTC15607 | 2008 | Bp* | BpKCTC |
| Producta | KBL988 | KCTC 13915BP | 2019 Aug. 16 | | BpYA44 |
| | KBL990 | KCTC 13917BP | 2019 Aug. 16 | | BpMG11 |
| | KBL991 | KCTC 13918BP | 2019 Aug. 16 | | BpMA68 |

| | | | |
|---|---|---|---|
| Enterococcus Faecium KBL986 | KCTC 13914BP | 2019 Aug. 16 | Ef |
| Blautia faecis KBL989 | KCTC 13916BP | 2019 Aug. 16 | Bf |
| Proteus terrae KBL985 | KCTC 13933BP | 2019 Sep. 3 | Pt |

*When referred to as Bp in the examples below, it refers to BpKCTC.

1-2. Clostridium difficile Culture and Generation of Spores

In order to culture Clostridium difficile strains for use in a CDI animal model and generate spores therefrom, Clostridium difficile VIP10463 strains were cultured on BHIS agar medium, and after activation through a total of two subcultures at 24-hour intervals, they were inoculated into BHIS broth and cultured for 24 hours. After adjusting an optical density (OD) value of cultured strains to be 0.2, 100 μl of each was plated on SMC agar medium and cultured at 37° C. for 7 days. Thereafter, all the cells were scraped off SMC agar, suspended in Cold PBS, stored at 4° C. for 24 hours, centrifuged at 14,000 rpm for 1 minute to remove supernatant, and then a process of re-suspending in Cold PBS was repeated twice. A pellet washed by repeating the above process was stored in 1 ml of 70% ethanol for 1 hour, centrifuged at 14,000 rpm for 1 minute to remove the supernatant, and then washed with Cold PBS twice. Then, it was suspended in 1 ml Cold PBS and stored at 4° C. until use.

### Example 2. Clostridium difficile Infection Inhibition Efficacy Evaluation in CDI Animal Model (1)—Each Strain was Administered 4 Times

2-1: CDI Animal Model Preparation and Strain Administration

In this Example, in order to determine whether a synergistic CDI inhibitory effect is achieved when each of Bp, Ef, Bf, and Pt is administered in combination with Cs, following experiments were conducted on an experimental group administered with each of Bp, Ef, Bf, Pt, and Cs single, a combination administration experimental group in which each of Bp, Ef, Bf, and Pt was administered in combination with Cs, a non-infected group and an infected group administered with PBS as control groups.

For the expression of CDI symptoms, C57BL/6 female 6-week-old mice were used. Mice introduced into an animal testing facility were subjected to a one-week acclimatization and stabilization period, and their body weights were measured to match a body weight average and standard deviation between groups. Eight mice were assigned to each group, and an experiment was repeated in two sets.

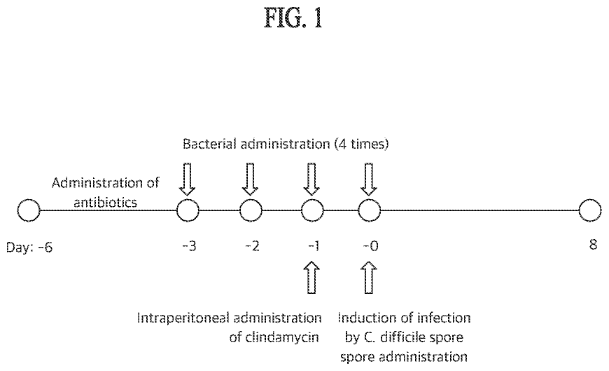

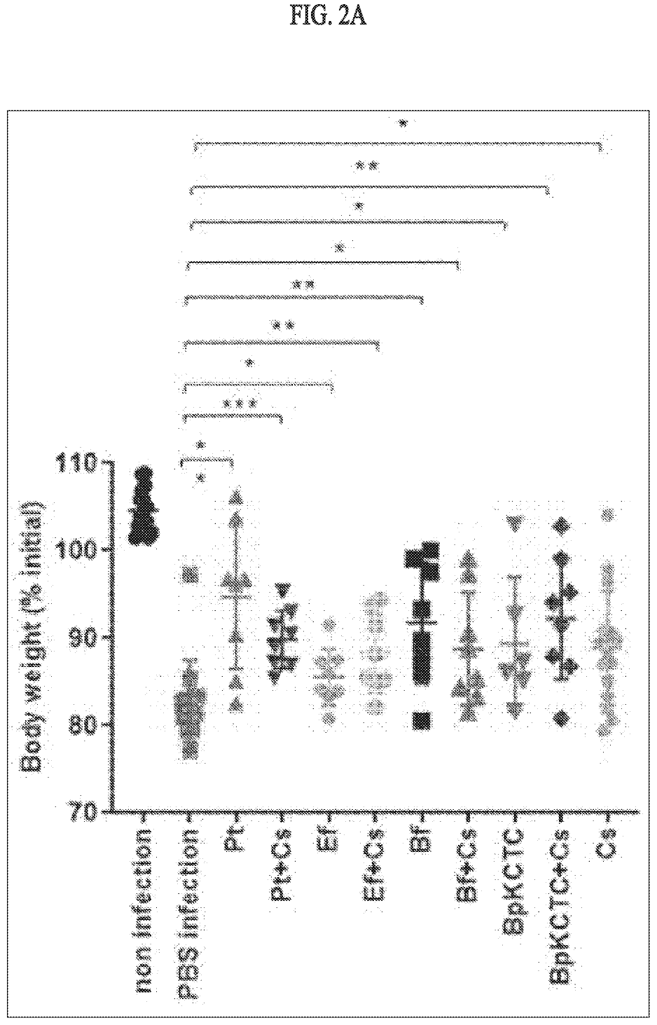

In the CDI prevention animal model (see FIG. 1), C57BL/6 mice in which intestinal microbial strains were disrupted by administering antibiotic cocktails (kanamycin (0.4 mg/ml), gentamicin (0.035 mg/ml), colistin (850 U/ml), metronidazole (0.215 mg/ml), and vancomycin (0.045 mg/ml)) was administered with drinking water for 3 days, the candidate strains of an experimental group were administered by oral zonde three times at 24-hour intervals from the third day after antibiotics were administered to the mice (1×10⁹ CFU/ml, 200 μl/mouse), and the candidate strains were administered once 12 hours after the third administration. Clindamycin was intraperitoneally administered 12 hours before the administration of the last candidate strain, 24 hours after administration of the last candidate strain, 10,000 spores of Clostridium difficile (C. difficile) obtained from Examples 1-2 above were suspended in 200 μl PBS and orally administered to experimental animals to infect C. difficile. The effects on CDI symptoms (weight change) and survival in mice of each experimental group and control group were measured every day for 8 days.

2-2: Result Analysis

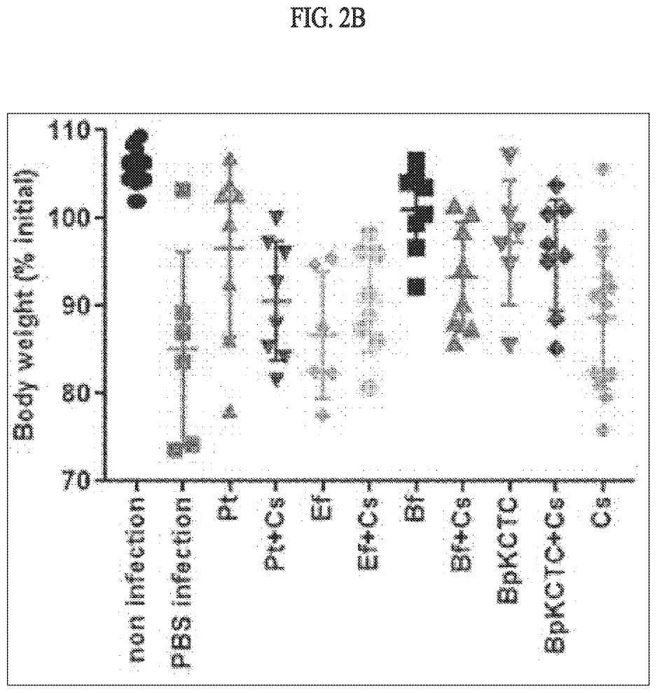

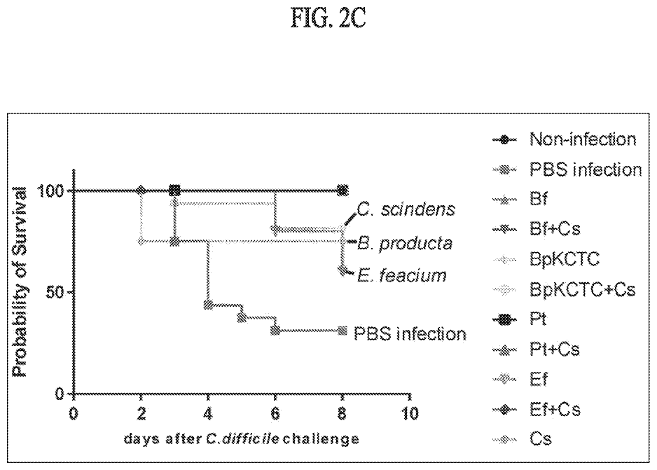

FIGS. 2a and 2b show the results of measuring body weight loss rate after 48 hours and 72 hours, respectively, after spore infection. Compared to a body weight change pattern in a control group (non infection) not infected with C. difficile, a body weight of the mice was significantly reduced in the control group (PBS infection) infected with C. difficile and administered only with PBS. In the case of Bp (BpKCTC) and Ef, compared to the single administration of Bp and Ef, body weight loss inhibition rate was significantly increased when Bp or Ef was administered in combination with Cs, and in the case of Bf and Pt, the single administration of Bf and Pt showed a better effect on the body weight loss inhibition rate than when the combination was administered with Cs, and this tendency was more clearly observed after 72 hours.

On the other hand, as a result of measuring a survival rate of mice infected with C. difficile, single administration groups of Cs, Bp and Ef showed a survival rate of about 60 to 80%, and it was found to have an effect of increasing the survival rate for CDI infection compared to the survival rate of mice in the control group administered only with PBS. In particular, a combined administration group of Ef and Cs and a combined administration group of Bp and Cs showed survival rate of 100%, confirming that the combined administration of Bp or Ef with Cs had an effect of increasing survival (FIG. 2C).

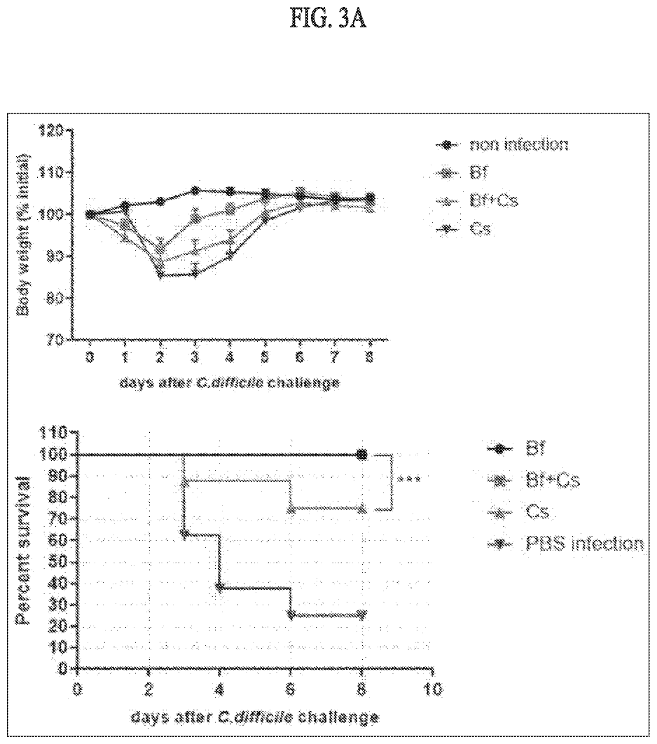

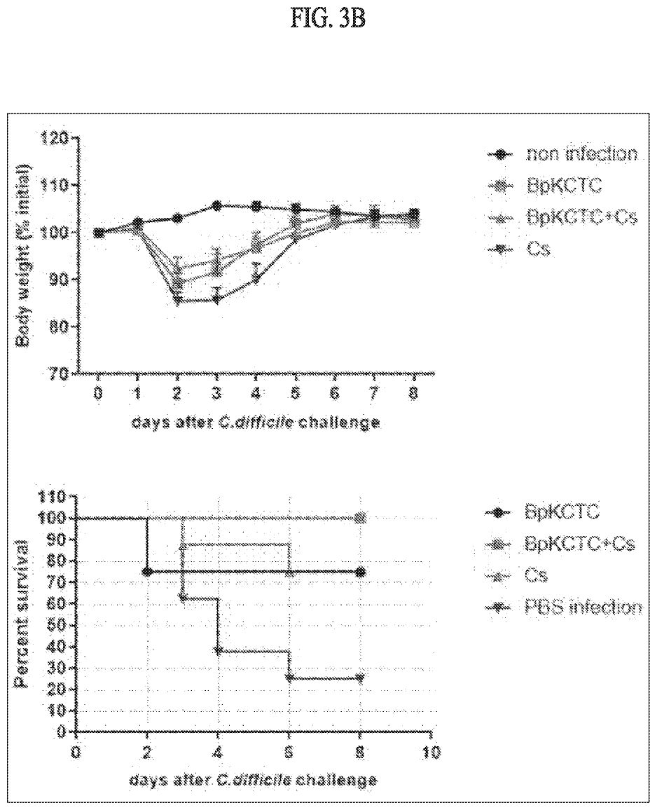

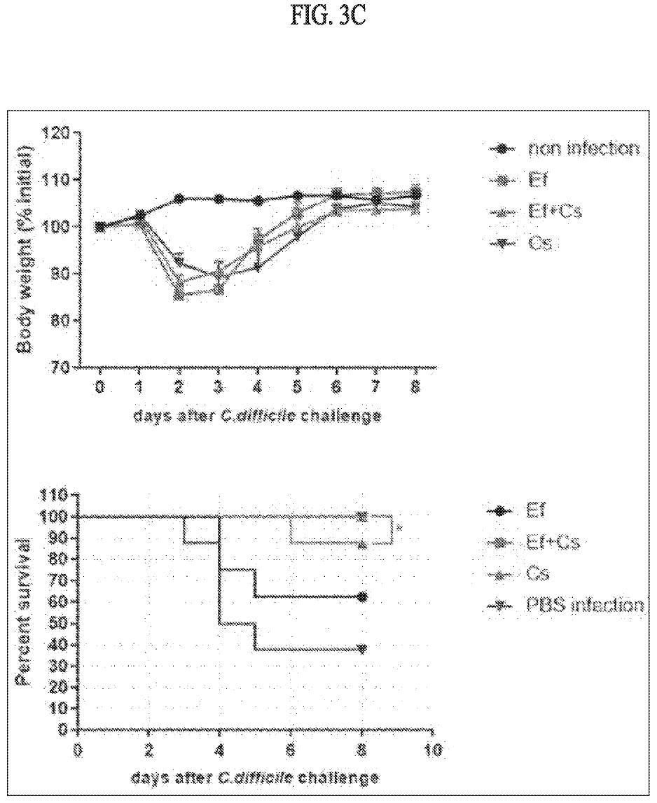

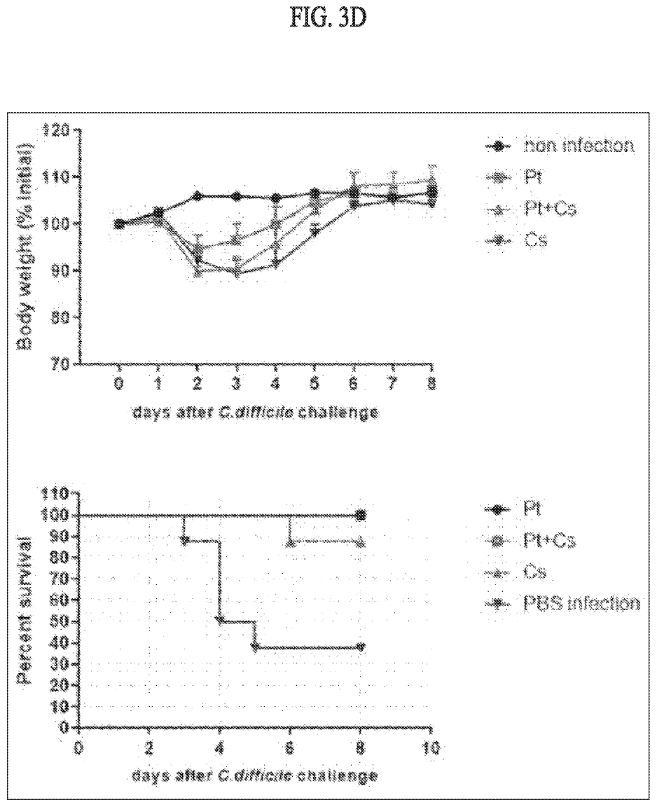

In addition, the body weight change and survival rate change test results were reorganized over time after C. difficile infection, and were shown in FIGS. 3A to 3D. As a result, even in the case of administration groups that showed body weight loss in the acute phase of C. difficile infection (about 2 to 5 days after infection), after the acute phase, it was found that a body weight of mice recovered to a level similar to that of a control mice not infected with C. difficile. In addition, in the case of survival rate, there was a difference in survival rate between the single or combination administration groups of each strain, but all of them maintained a higher survival rate than a PBS administration group. In particular, in the case of Bp and Ef, in the group administered in combination with Cs, and in the case of Bf and Pt, in the single administration group, an inhibition rate and survival rate of body weight loss in the acute phase of infection (about 2 to 5 days after infection) were excellent (see FIG. 3). The average minimum body weights of single administration groups of Bf and Pt and combination administration groups of Bp or Ef with Cs shown in FIG. 3 are as follows:

TABLE 2

| | Bf alone | | | Pt alone |
|---|---|---|---|---|
| | administration | Bp + Cs | Ef + Cs | administration |
| Repetition set/n | 2 sets/8 | 2 sets/8 | 2 sets/8 | 2 sets/8 |
| Average minimum | 88.7914%(p.i. 2) | 92.296%(p.i. 2) | 85.532%(p.i. 2) | 94.722%(p.i. 2) |
| body weight | | | | |
| Death rate | 0% | 0% | 0% | 0% |

* p.i. means days post infection.

### Example 3. Clostridium difficile Infection Inhibition Efficacy Evaluation in CDI Animal Model (2)—Each Strain was Administered 2 Times

3-1: CDI Animal Model Preparation and Strain Administration

When Bp, Ef, Bf, Pt, and Cs strains were administered alone or in combination, a preventive effect against CDI was observed again in an animal model different from Example 2.

C57BL/6 female 6-week-old mice were used for the expression of CDI symptoms, and 4 mice were assigned to each group. Mice introduced into the animal testing facility were subjected to a one-week acclimatization and stabilization period, and their body weights were measured to match the body weight average and standard deviation between groups.

Antibiotic cocktail (kanamycin (0.4 mg/ml), gentamicin (0.035 mg/ml), colistin (850 U/ml), metronidazole (0.215 mg/ml), and vancomycin (0.045 mg/ml) 6 days before administration of C. difficile spores was provided as drinking water to the animal model for 3 days, and after replacement with normal drinking water, it was stabilized for 2 days.

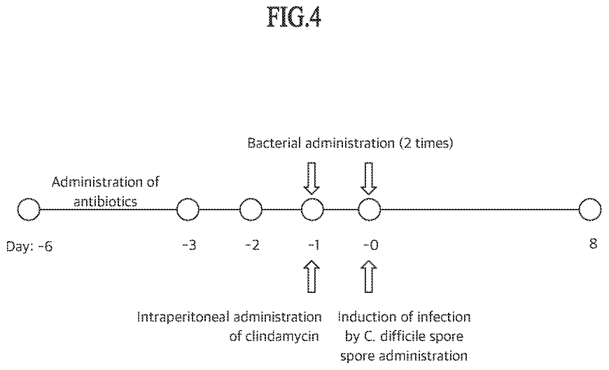

To the stabilized animal model, immediately after intraperitoneal administration of Clindamycin (10 mg/kg) 24 hours before administration of C. difficile spores, candidate strains were orally administered (1×10⁹ CFU/ml, 200 μl/mouse), and candidate strains were administered once more 12 hours before spore infection. 12 hours after the administration of last candidate strains, 10,000 C. difficile spores obtained from Examples 1-2 were suspended in 200 μl PBS and orally administered to experimental animals to induce infection. After measuring the body weight at the time of infection with C. difficile spores, the body weight and the dead individuals were measured at 24-hour intervals to confirm the efficacy of inhibiting CDI infection (FIG. 4).

The above experiment was repeated twice, and in the second experiment, the group administered Bp, Ef, Pt, and Bf in combination with Cs was also tested.

3-2: Result Analysis

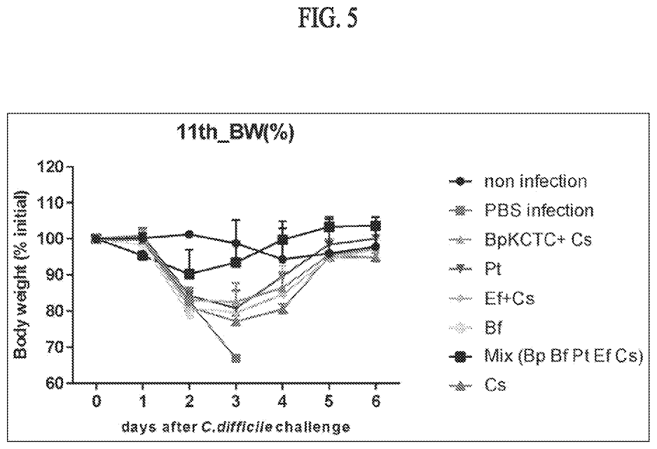

An average lowest body weight and death rate of each candidate strain administration group measured in each of two experiments are shown in Tables 3 and 4, respectively, and body weight change over time after infection with C. difficile is shown in FIG. 5.

TABLE 3

| | Bf alone | | | Pt alone |
|---|---|---|---|---|
| | administration | Bp + Cs | Ef + Cs | administration |
| Repetition sets/n | 1 set/4 | 1 set/4 | 1 set/4 | 1 set/4 |
| Average minimum | 85.5882%/p.i. 3 | 90.3797%/p.i. 2 | 91.3861%/p.i. 2 | 87.40408%/p.i. 3 |
| body weight | | | | |
| Death rate | 0% | 0% | 0% | 0% |

TABLE 4

| | Pt alone | | | Pt alone | Bp + Ef + Bf + |
|---|---|---|---|---|---|
| | administration | Bp + Cs | Ef + Cs | administration | Pt + Cs |
| Repetition sets/n | 1 set/4 | 1 set/4 | 1 set/4 | 1 set/4 | 1 set/4 |
| Average minimum | — | 81.6312%/p.i. 3 | 79.5231%/p.i. 3 | 80.8238%/p.i. 3 | 90.2471%/p.i. 2 |
| body weight | | | | | |
| Death rate | 100% | 0% | 25% | 50% | 0% |

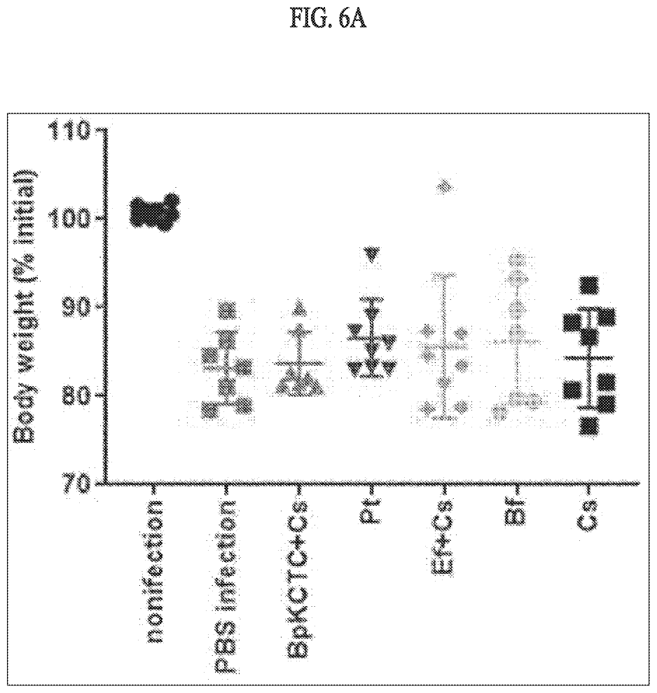

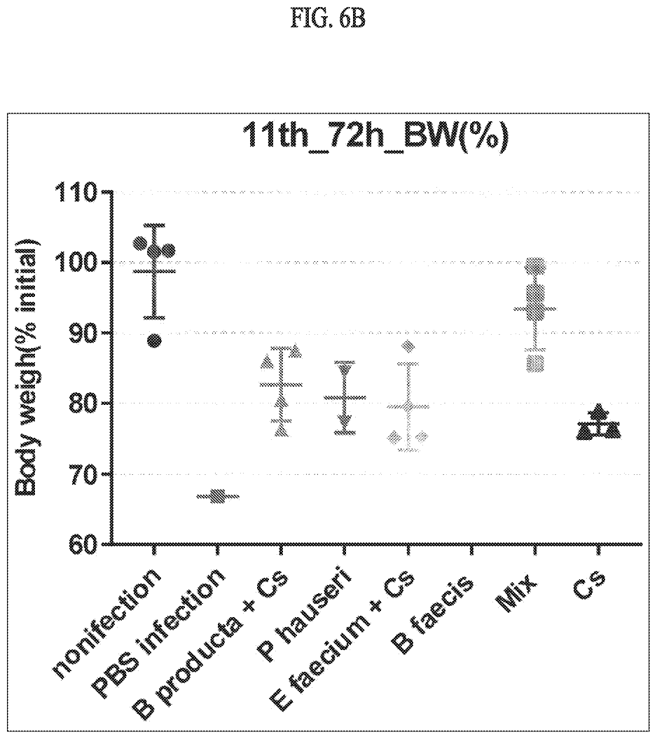

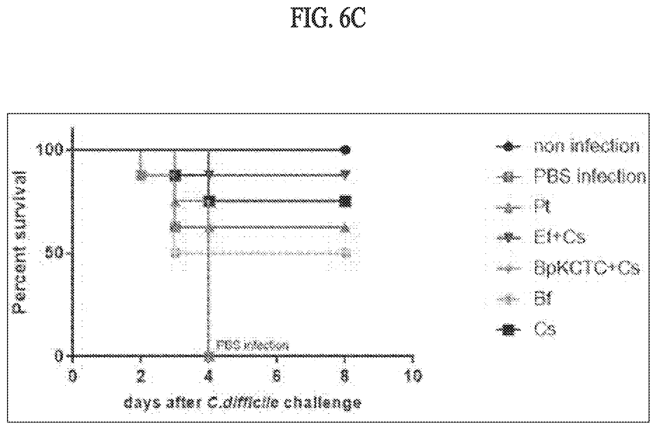

As confirmed in Table 4, in the second experiment, a Cs, Bp, Ef, Bf, and Pt combination administration group showed the most excellent effect of suppressing weight loss and improving survival rate. When examining the body weight change rate over time after spore infection, group in which all the five strains were administered in combination showed a superior body weight loss inhibition rate compared to the other groups (see FIG. 5). On the other hand, results of two experiments measuring the body weight change and survival rate 48 hours and 72 hours after CDI induction (except for experimental results of the Cs, Bp, Ef, Pt, and Bf combination administration group in the second experiment) are shown in FIGS. 6A to 6C. Looking at results shown in FIGS. 6A to 6C, a Bp and Cs combination administration group and an Ef and Cs combination administration group showed a tendency to suppress weight loss and improve survival rate compared to a Cs single administration group, but a Bf single administration group and a Pt single administration group were inferior to a Cs single administration group in terms of survival rate improvement. In experimental results shown in FIGS. 6A to 6C, there was no significant difference in effects of each group as a whole, while one reason is considered to be that time required for intestinal colonization of each strain was reduced as a animal model was changed.

### Example 4. Identification of Key Strains Exhibiting Synergistic Effects Upon Combination Administration

### Example 4-1. Confirm the Contribution of Each Strain to the Synergistic Effects

In a combination of combined administration of all 5 strains (Cs, Bp, Ef, Bf and Pt) confirmed to exhibit the best effect in Example 3, CDI inhibitory efficacy of five groups of combined administration of 4 strains, each excluding one strain from the 5 strains combination, was evaluated using the same animal model as that used in Example 3.

With three control groups: a non-infection group, a PBS infection group, and Cs single administration group, experiments were repeated twice with 4 animals per group for 5 strains combination (combination of Cs, Bp, Ef, Bf and Pt; Mix) and 4 strains combinations excluding one strain from each of the 5 strains combination (Mix-Cs, Mix-Bp, Mix-Ef, Mix-Bf and Mix-Pt) (9 groups in total). In the experimental groups, strains were administered at a concentration of 1×10⁹ CFU/ml.

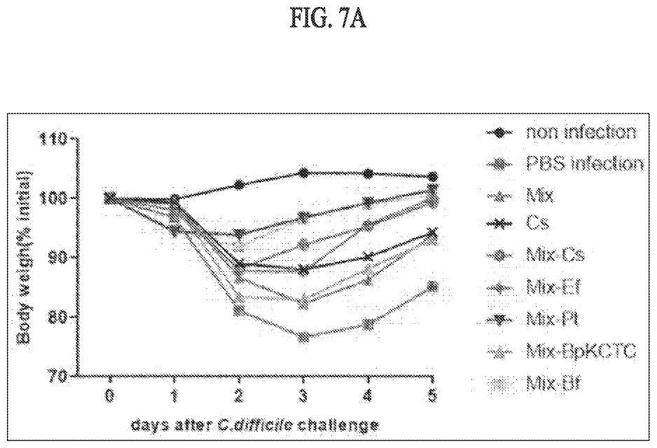

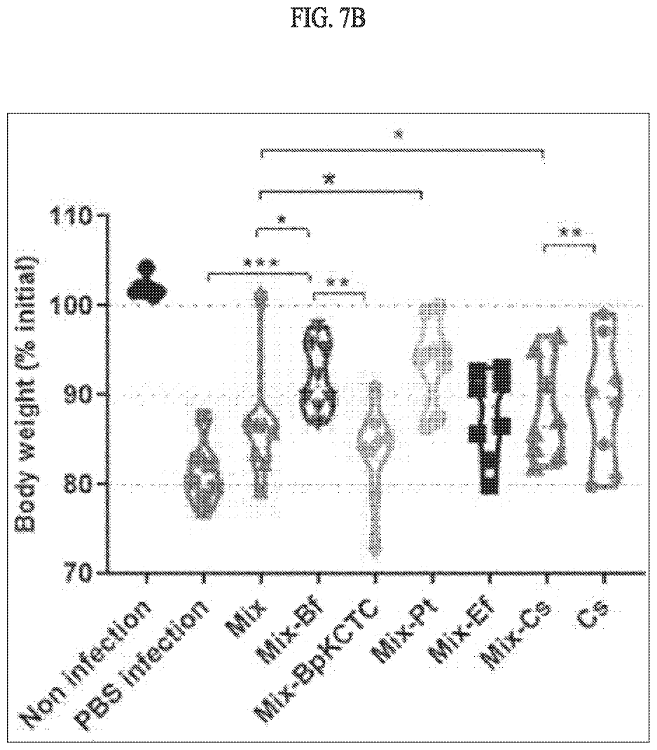

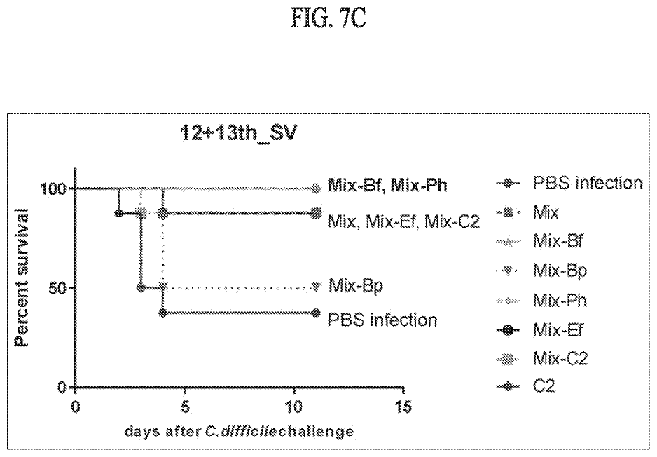

Results of measuring body weight loss rate and survival rate for 3 control groups and 6 experimental groups are shown in FIGS. 7A to 7C.

As confirmed from FIGS. 7A to 7C, when Cs, Bp or Ef was excluded from the combination, the CDI inhibitory effect was significantly reduced, and they were found to be strains that contributes to a synergistic CDI inhibitory effect when administered in combination with other strains. Through these results, it was reconfirmed that Cs, Bp and Bf play an important role in exerting the CDI inhibitory effect, and as confirmed in Example 3, Bp and Ef exhibit a synergistic CDI inhibitory effect when used in combination with Cs.

### Example 4-2. Confirmation of CDI Inhibitory Effect According to Strain Dosage Change

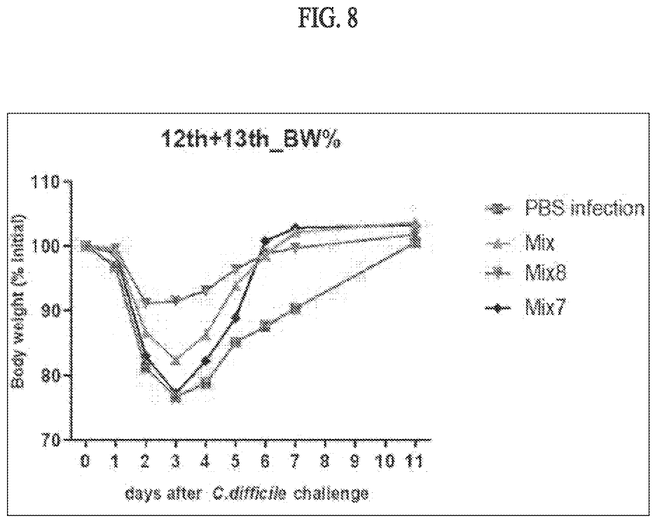

In addition, in the same manner as in Example 3, CDI inhibitory efficacy changes were confirmed by varying the total cell administration concentrations of the 5 strains combination (Cs, Bp, Ef, Bf and Pt) to 1×10⁷ CFU/ml (Mix7 experimental group), 1×10⁸ CFU/ml (Mix8 experimental group), 1×10⁹ CFU/ml (Mix9 experimental group), respectively. Results of measuring body weight changes after inducing CDI are shown in FIG. 8.

As shown in FIG. 8, when CDI is induced, body weight of a control group (an infected group administered with PBS) is generally reduced by about 20%. On the other hand, among the three experimental groups in which 5 strains were administered but the strain concentrations were different, the experimental groups administered with 5 strains at 1×10⁹ CFU/ml and 1×10⁸ CFU/ml (Mix9 and Mix8 experimental groups, respectively) showed that the weight loss rate was significantly reduced compared to the control group. However, when the concentration was 1×10⁷ CFU/ml (Mix7 experimental group), there was no significant difference from a control group in terms of body weight loss inhibition. Therefore, it was confirmed that a more excellent CDI inhibitory effect appeared when the administration concentration of 5 strains was 1×10⁸ CFU/ml or more.

### Example 5. Selection of Optimal Strain Combination Showing Elevated C. difficile Infection Inhibitory Effect

In the above-described example, since the synergistic CDI inhibitory effect was confirmed when Bp and Ef were used in combination with Cs, in this example, based on the combination of these three strains, it was confirmed whether the CDI inhibitory effect can be further increased when Pt or Bf is additionally combined. In addition, experiments were conducted using four types of Bp strains (BpKCTC, BpMG11, BpYA44, BpMA68) in order to confirm whether an equivalent CDI inhibitory effect could be achieved even when different strains belonging to the same species were used in the strain combination of the present disclosure.

The same animal model as in Example 3 is used, a total of 9 groups were tested twice: a non-infected group, a PBS infected group, a Cs single administration group, Cs+Bf+Ef group, Cs+Bp+Ef group, Cs+Ef+Bf with any one of BpKCTC, BpMG11, BpYA44 and BpMA68 groups.

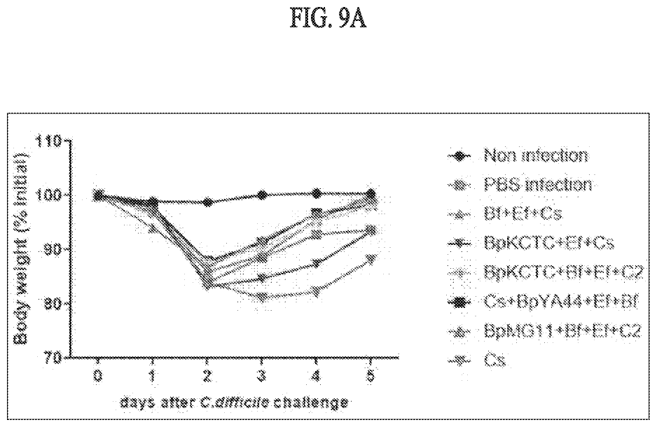

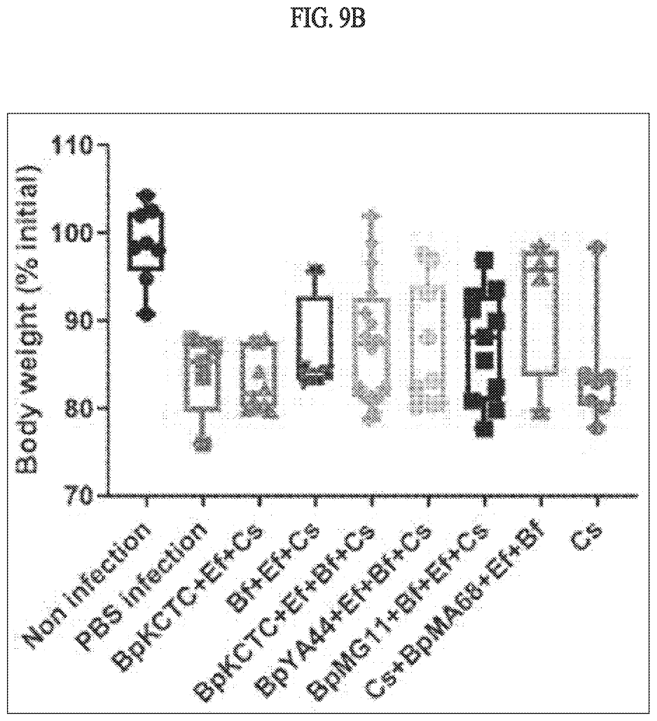

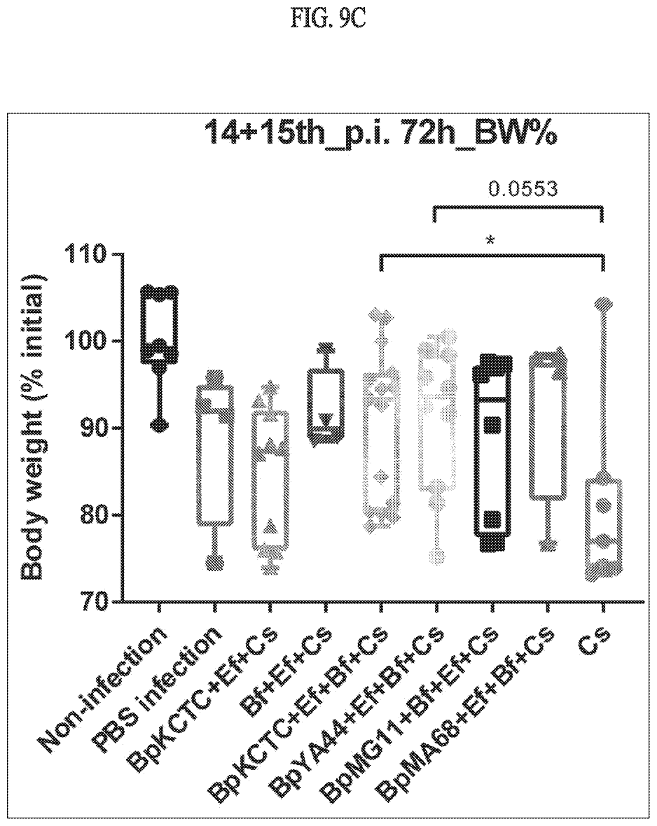

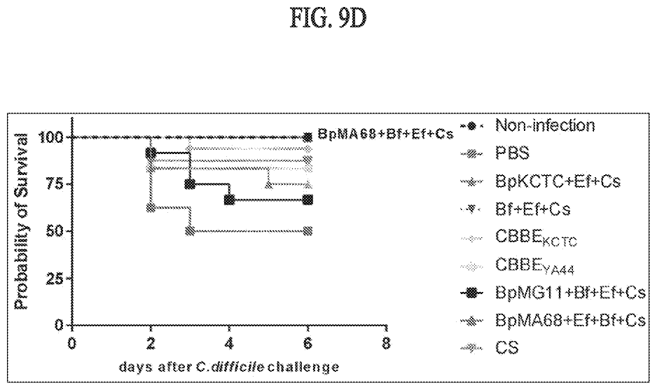

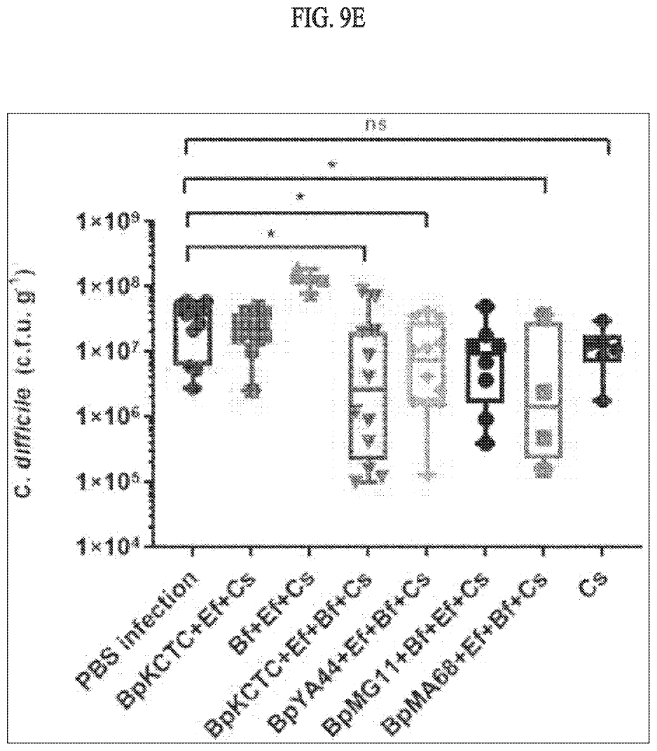

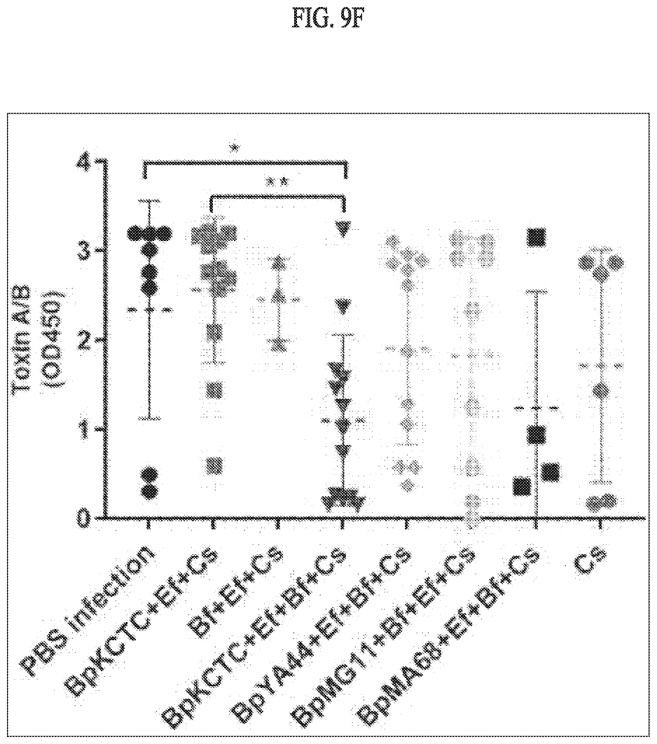

The results of measuring body weight change over time, body weight change after 48 and 72 hours of CDI induction, survival rate, stool c.f.u. after 24 hours, and stool toxin after 24 hours for each experimental group are shown in FIGS. 9A to 9F.

Considering the results of FIGS. 9A to 9F, the combination of 4 strains of Cs+Bp+Ef+Bf showed excellent effects in terms of weight loss inhibition and survival rate compared to Cs single administration or the combinations of 3 strains of Cs+Bp+Ef or Cs+Bp+Bf, and also significantly reduced stool c.f.u. and stool toxin 24 hours after induction of C. difficile infection. In addition, it was found that these effects of the present disclosure are commonly observed when BpKCTC, BpMA66, BpYA44 and BpMG11 are used as Bp strains, so that the CDI infection inhibitory effect can be achieved using various strains belonging to each species in the strain combination according to the present disclosure.

### Example 6. Reconfirmation of Synergistic Effect of Optimal Strain Combination (Cs, Bp, Bf and Ef)

A synergistic CDI inhibitory effect according to the combination of Cs, Bp, Bf and Ef strains, which is the optimal strain combination that showed an excellent CDI inhibitory effect in Example 5, was confirmed once again.

First, an effect difference between the case of administering the Cs+Bp+Bf+Ef combination and the case of administering Cs strain alone was compared.

In addition, differences in effects according to the case of administering a Cs+Bp+Bf+Ef combination, the case of administering a Cs+Bp+Bf+Ef+Pt combination in which Pt was added to the above combination, or the case of administering the Cs+Bp+Pt+Ef combination in which Pt was added instead of Bf were compared. Furthermore, the difference in effect from the case of administration of the 3 strains Cs+Bp+Ef combination was compared.

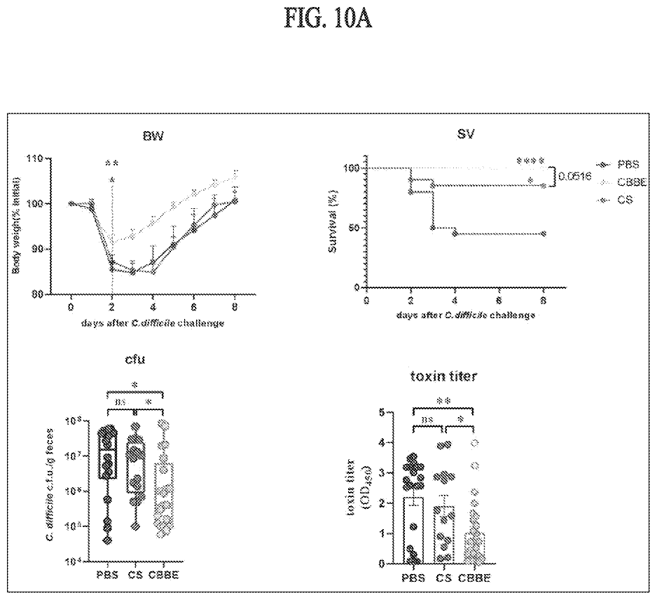

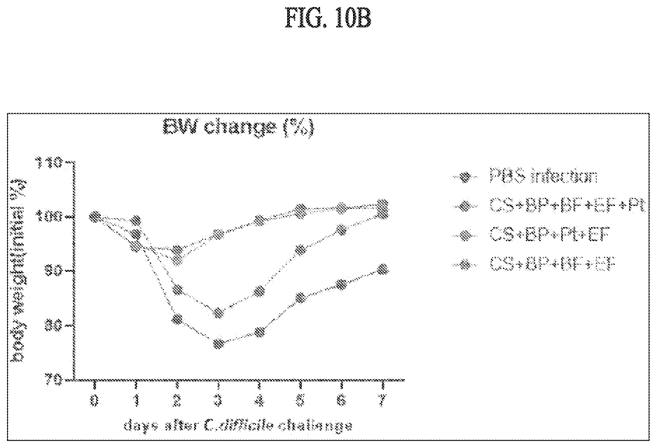

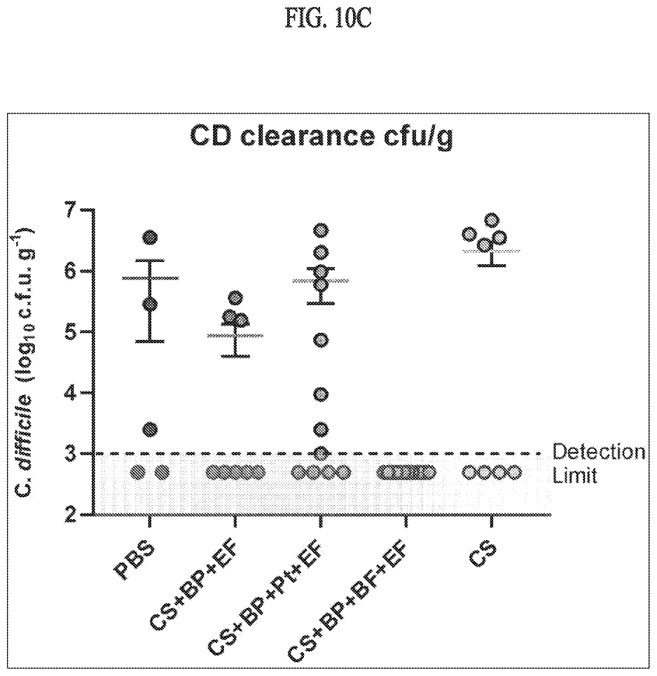

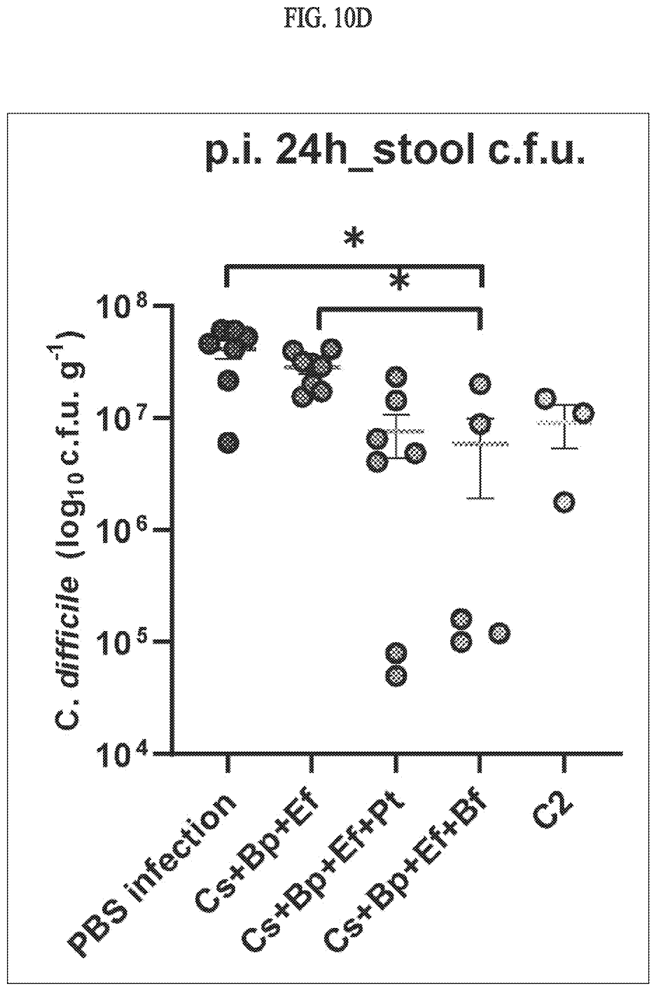

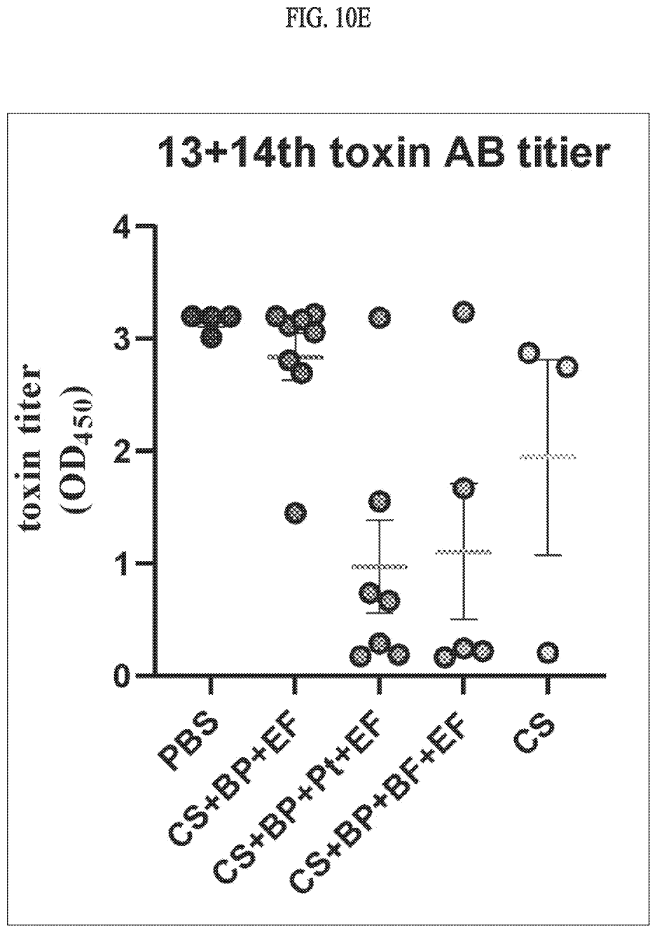

The same animal model as in Example 3 was used, but a PBS infection group, a Cs+Bp+Bf+Ef administration group, a Cs single administration group, a Cs+Bp+Bf+Ef+Pt administration group, a Cs+Bp+Pt+Ef administration group, and a Cs+Bp+Ef administration group were tested twice, and results of measuring weight change over time, survival rate, c.f.u. of C. difficile, stool c.f.u. after 24 hours, and stool toxin after 24 hours in each experimental group are shown in FIGS. 10A to 10E.

Considering results of FIGS. 10A to 10E, it was confirmed that the combination of 4 strains of Cs+Bp+Bf+Ef showed excellent effects in terms of weight loss inhibition and survival rate compared to the case of administering the Cs strain alone or administering the Cs+Bp+Bf+Ef+Pt combination, the Cs+Bp+Pt+Ef combination, or the Cs+Bp+Ef combination, and also significantly reduced stool c.f.u. and stool toxin 24 hours after induction of C. difficile infection.

In particular, in the case of the combination of 4 strains of Cs+Bp+Bf+Ef, an effect of inhibiting body weight loss was more excellent even when compared to the case of administration with a combination in which Pt was added (FIG. 10B). Therefore, it was confirmed that the administration of more strains exhibiting the CDI infection inhibitory effect does not necessarily show a better effect, and it was confirmed that among various strain combinations, it can be expected that the combination of Cs+Bp+Bf+Ef will have the best effect.

In addition, when 4 strains were administered in combination, such as the Cs+Bp+Bf+Ef combination, it was confirmed that stool c.f.u. and stool toxin reduction effects were also measured to be very significant compared to the case of administration of 3 strains in combination with Cs+Bp+Ef, so it was confirmed that the 4 strains combination had a better inhibitory effect on CDI infection by suppressing C. difficile colonization or intestinal toxin production after infection.

### Example 7. Ex Vivo Experiments of Optimal Strain Combination (Cs, Bp, Bf and Ef)

CDI inhibitory effects of the Cs, Bp, Bf and Ef strain combination, which is an optimal strain combination that showed an excellent CDI inhibitory effect in Example 5, was confirmed through ex vivo experiment.

The same animal model as in Example 3 was used, but 24 hours after administration of Clindamycin, the mouse was opened and a cecum portion was separated and weight was measured. In addition, it was diluted with PBS to a concentration of 5% to 10% (W/V), and then 1 ml of a 1% culture solution isolated from the cecum was dispensed into a 14 ml round bottom tube. Both a control group and experimental groups were inoculated with C. difficile at 1×10⁷ CFU/ml, and the experimental groups were inoculated with the strains of the present disclosure at 1×10⁷ CFU/ml. The experimental groups were inoculated with Cs strain alone or with various combinations of strains, and the control group was inoculated with PBS and they were cultured at 37° C. for 36 hours. Specifically, the experimental groups consisted of Cs+Bp+Bf+Ef (CBBE), Bp+Bf+Ef (ΔCS), Cs+Bf+Ef (ΔBP), Cs+Bp+Ef (ΔBF), Cs+Bp+Bf (ΔEF) combinations. The cultures were centrifuged to extract bacterial DNA from precipitates, and a pH of the supernatant was measured. The isolated bacterial DNA was quantified using species-specific primers for each strain (Clostridium difficile, Clostridium scindens, Blautia producta, Blautia faecis species-specific primers, SEQ ID NO: 9 to SEQ ID NO: 16).

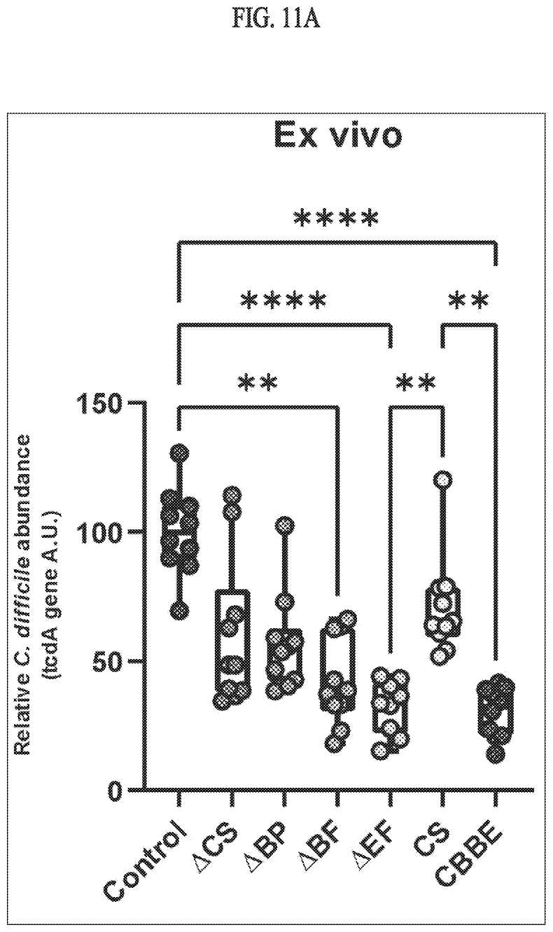

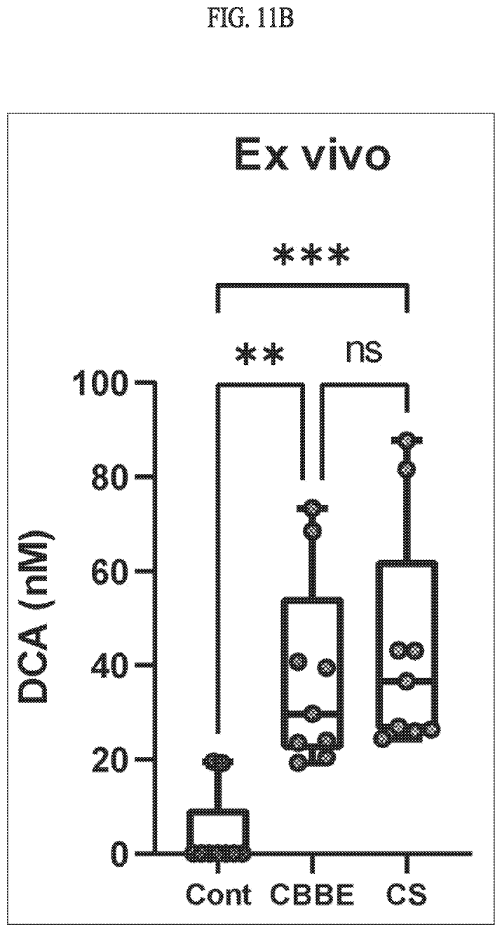

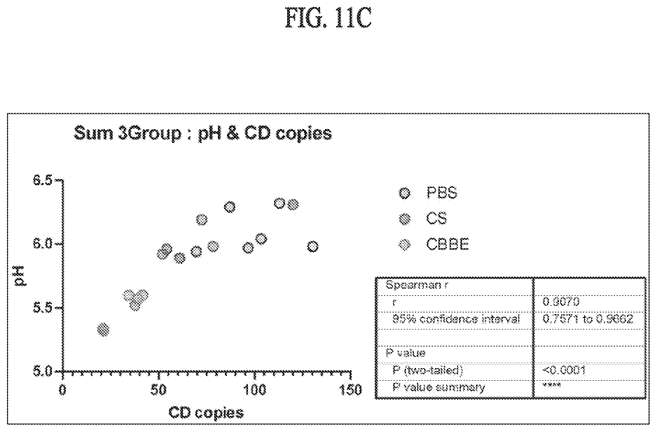

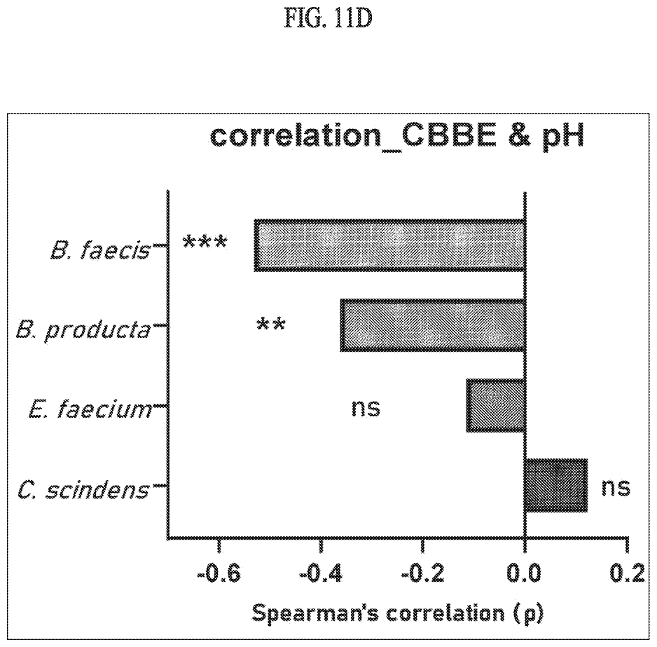

In FIG. 11A, a relative number of C. difficile according to a combination of each of strains was measured and shown, and in all experimental groups, the reduction effect of C. difficile was shown compared to a control group, and in particular, a combination of Cs+Bp+Bf+Ef (CBBE) showed the most excellent effect. On the other hand, as a result of measuring and comparing a concentration of a secondary bile acid (deoxycholic acid; DCA), which has an inhibitory effect on CDI infection in ex vivo samples, the value of the Cs+Bp+Bf+Ef (CBBE) combination and Cs single inoculation were similarly measured, confirming that the efficacy of inhibiting CDI infection was excellent in both groups (FIG. 11B).

As a result of pH analysis of a centrifuged supernatant, the pH in the sample was low in an experimental group inoculated with the Cs+Bp+Bf+Ef (CBBE) combination, and it was confirmed that CDI infection inhibitory effect by the strain combination was caused by a factor caused by deoxycholic acid and a factor lowering the pH (FIG. 11C). In addition, as a result of analyzing correlation by measuring the number of each strain in a sample of Cs+Bp+Bf+Ef (CBBE) and comparing it with pH and the number of C. difficile strains, it was confirmed that role of lowering the pH of the sample was caused by Bp and Bf, and it was confirmed that the Bp, Bf and Cs strains were involved in the growth inhibition of C. difficile. (FIGS. 11D and 11E). Therefore, it was confirmed that the combination of the strains can exhibit a synergistic effect with respect to inhibition of CDI infection through production of bile acids and decrease in pH.

### Example 8. A Strain Combination of the Present Disclosure does not Affect an Immune System

It is known that CD4⁺ T cells express the transcription factor Foxp3 and play an important role in maintaining immunological homeostasis. It has been reported that a large number of Foxp3-expressing cells are present in large intestine, and only Treg cells localized in the large intestine constantly express high levels of IL-10, an immunosuppressive cytokine. Accordingly, attempts have been made to prevent or treat autoimmune diseases, inflammatory diseases, and various bacterial infections by suppressing excessive inflammation caused by immunity by inducing Treg cells (European Patent No. 2 575 835).

In this Example, in order to confirm whether the C. difficile infection inhibitory effect of the strain combination of the present disclosure is related to induction of Treg cells, four groups were tested, including an antibiotic-untreated group (ABX(−)), an antibiotic treatment+PBS administration group (ABX(+) PBS), an antibiotic treatment+the strain combination of the present disclosure administration group (ABX(+) MIX), and an antibiotic treatment+positive control strain administration group (ABX(+) KBL693), and 4 mice were assigned to each group. As the strain combination of the present disclosure, Ef (KBL986)+Cs (KBL987)+Bp (KBL988)+Bf (KBL989) was administered, and a dosage was 1 to 5×10⁸ CFU/200 μl per mouse by day 3, and 1 to 5×10⁹ CFU/200 μl on day 4 and day 5. In addition, as a positive control strain, Lactobacillus crispatus KBL693 (date of deposit: 2018 Apr. 27, deposit number: KCTC 13519BP) was administered.

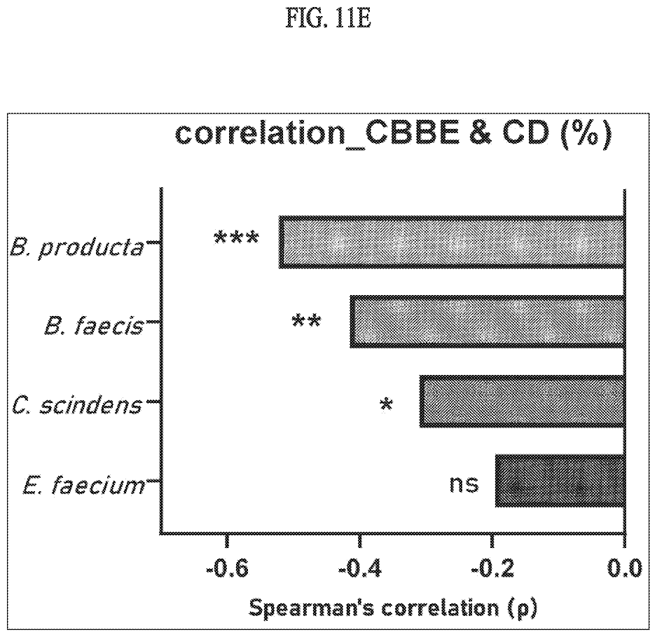

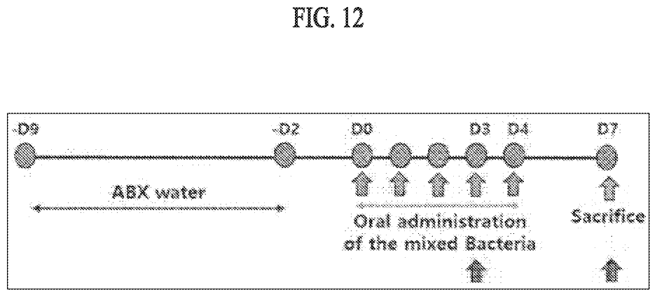

In the 3 groups excluding the antibiotic untreated group, mice were treated with antibiotics (ABX) for 9 days to remove intestinal microorganisms, and after stabilization for 3 days, PBS, the strain combination of the present disclosure or the positive control strain was administered once daily from day 3, and administered for 5 days. Treg cells and iTreg cells of colonic LP, siLP, and mesenteric LNs were measured in mice sacrificed after 3 days of administration (D3), and mice sacrificed 2 days after 5 days of administration (D7) (FIG. 12).

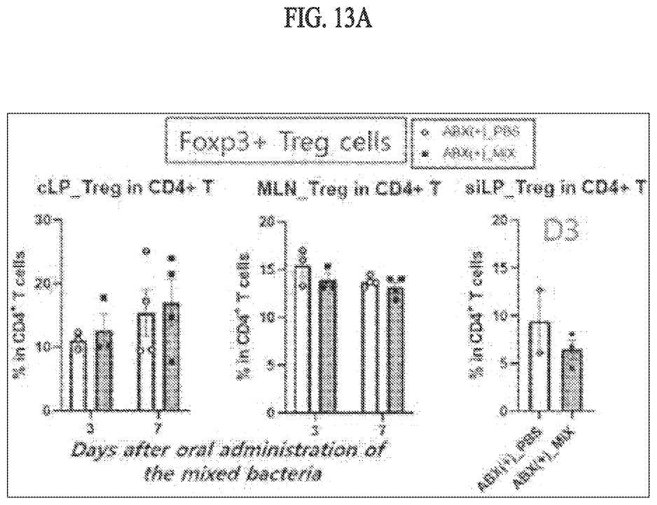

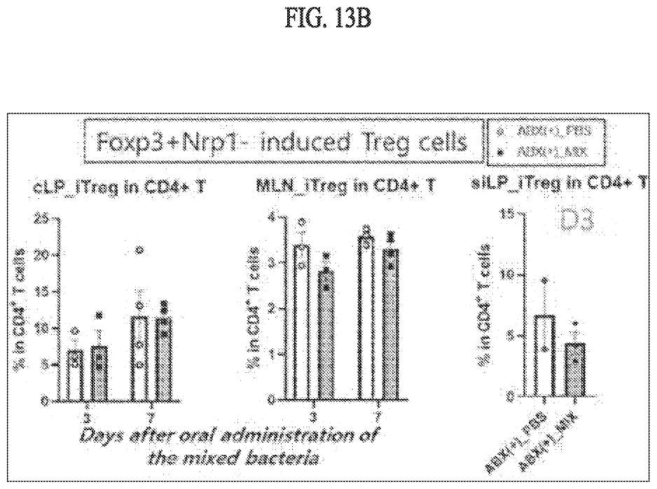

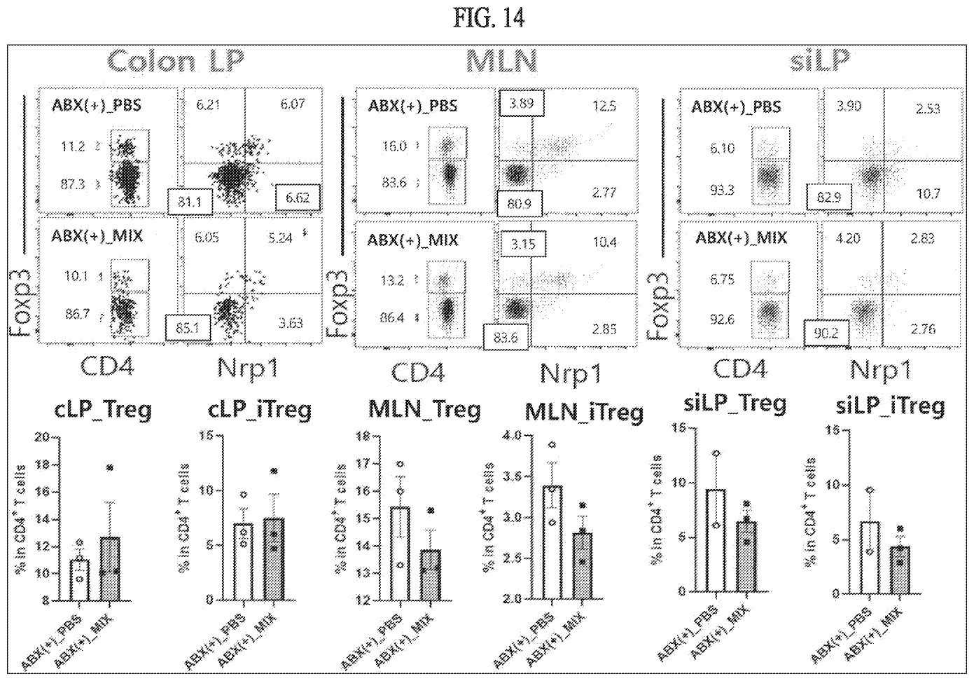

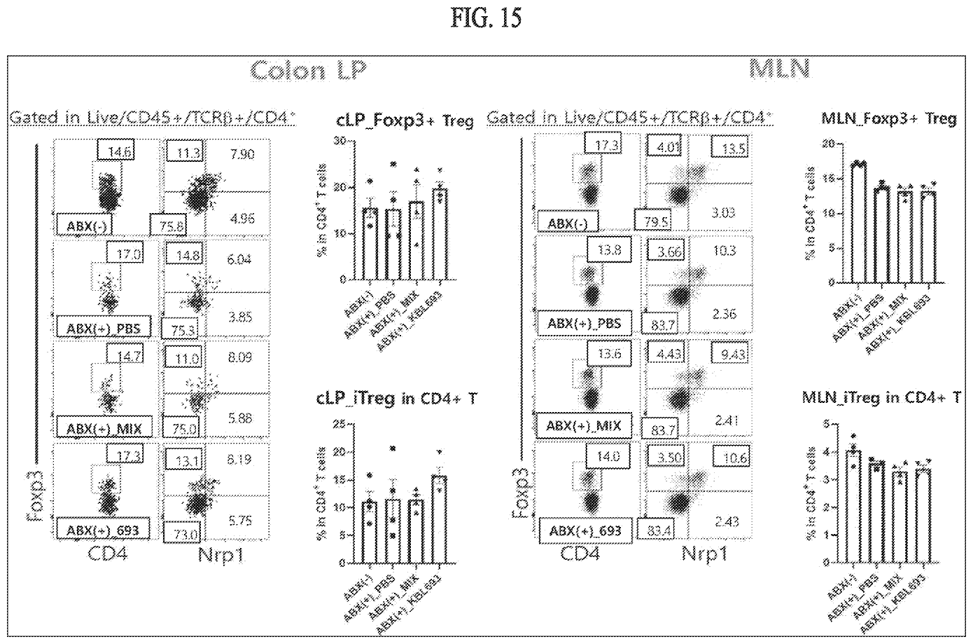

As a result of the measurement, colonic Treg cells were increased at D7 rather than D3, confirming that the antibiotic treatment was properly performed (FIG. 13A). The group administered with the strain combination of the present disclosure showed levels of colonic Treg cells and iTreg cells similar to those of a group administered with PBS, indicating that increase in Treg cells was not induced (FIG. 13). Conversely, in the KBL693 administered strain, which is a positive control strain, a significant increase in Foxp3+Treg was shown (see a left figure of FIG. 15). On the other hand, the siLP and mesenteric LNs were not affected by antibiotic treatment and administration of the strain combination of the present disclosure (FIGS. 13B, 14, and 15).

Having described specific embodiments of the present disclosure in detail above, it will be clear to those skilled in the art that these specific descriptions are only preferred embodiments, and the scope of the present disclosure will be not limited thereby. Accordingly, it will be said that the substantial protection scope of the present disclosure is defined by the appended claims and their equivalents.

### [Deposit Number]

Depository Institution Name: Korean Collection for Type Culture

Deposit number: KCTC13277BP

Deposit Date: 2017 May 29

Depository Institution Name: Korean Collection for Type Culture

Deposit number: KCTC13915BP

Deposit Date: 2019 Aug. 16

Depository Institution Name: Korean Collection for Type Culture

Deposit number: KCTC13917BP

Deposit Date: 2019 Aug. 16

Depository Institution Name: Korean Collection for Type Culture

Deposit number: KCTC13918BP

Deposit Date: 2019 Aug. 16

Depository Institution Name: Korean Collection for Type Culture

Deposit number: KCTC13914BP

Deposit Date: 2019 Aug. 16

Depository Institution Name: Korean Collection for Type Culture

Deposit number: KCTC13916BP

Deposit Date: 2019 Aug. 16

Depository Institution Name: Korean Collection for Type Culture

Deposit number: KCTC13933BP

Deposit Date: 2019 Sep. 3

1. A pharmaceutical composition for preventing or treating Clostridium difficile infection, comprising one or more selected from the group consisting of cells, cultures, lysates and extracts of Clostridium scindens, Blautia producta and Enterococcus faecium.

2. The pharmaceutical composition of claim 1, wherein the Clostridium scindens has a 16s rDNA sequence that is 97% or more identical to SEQ ID NO: 1, the Blautia producta has a 16s rDNA sequence that is 97% or more identical to any one of SEQ ID NOs: 2 to 5, and the Enterococcus faecium has a 16s rDNA sequence that is 97% or more identical to SEQ ID NO: 6.

3. The pharmaceutical composition of claim 1, wherein the Clostridium scindens is Clostridium scindens KBL987 strain with deposit number KCTC13277BP, the Blautia producta is Blautia producta ATCC27340 strain with deposit number KCTC15607, Blautia producta KBL988 strain with deposit number KCTC13915BP, Blautia producta KBL990 strain with deposit number KCTC13917BP or Blautia producta KBL991 strain with deposit number KCTC13918BP, and the Enterococcus faecium is Enterococcus faecium KBL986 strain with deposit number KCTC13914BP.

4. The pharmaceutical composition of claim 1, further comprising one or more selected from the group consisting of cells, cultures, lysates, and extracts of one or more of Blautia faecis and Proteus terrae.

5. The pharmaceutical composition of claim 4, wherein the Blautia faecis has a 16s rDNA sequence that is 97% or more identical to SEQ ID NO: 7, and the Proteus terrae has a 16s rDNA sequence that is 97% or more identical to SEQ ID NO: 8.

6. The pharmaceutical composition of claim 4, wherein the Blautia faecis is Blautia faecis KBL989 strain with deposit number KCTC13916BP, and the Proteus terrae is Proteus terrae KBL985 strain with deposit number KCTC13933BP.

7. The pharmaceutical composition of claim 4, further comprising one or more selected from the group consisting of cells, cultures, lysates, and extracts of Blautia faecis and Proteus terrae.

8. The pharmaceutical composition of claim 1, comprising 1×10⁸ CFU/ml or more of cells.

9. A food composition for preventing or improving Clostridium difficile infection, comprising one or more selected from the group consisting of cells, cultures, lysates, and extracts of Clostridium scindens, Blautia producta, and Enterococcus faecium.

10. The food composition of claim 9, further comprising one or more selected from the group consisting of cells, cultures, lysates, and extracts of one or more of Blautia faecis and Proteus terrae.

11. The food composition of claim 9, further comprising one or more selected from the group consisting of cells, cultures, lysates, and extracts of Blautia faecis and Proteus terrae.

12. The food composition of claim 9, comprising 1×10⁸ CFU/ml or more of cells.


SEQUENCE LISTING (16 sequences)

SEQ ID NO: 1 - DNA, 1463 bp, Artificial Sequence
  Clostridium scindens
ctcaggatgaacgctggcggcgtgcctaacacatgcaagtcgaacgaagcgcctggccccgacttcttcggaacgaggagccttgcgactgagtggcggacgggtgagtaacgcgtgggcaacctgccttgcactgggggataacagccagaaatggctgctaataccgcataagaccgaagcgccgcatggcgcggcggccaaagccccggcggtgcaagatgggcccgcgtctgattaggtagttggcggggtaacggcccaccaagccgacgatcagtagccgacctgagagggtgaccggccacattgggactgagacacggcccagactcctacgggaggcagcagtggggaatattgcacaatgggggaaaccctgatgcagcgacgccgcgtgaaggatgaagtatttcggtatgtaaacttctatcagcagggaagaagatgacggtacctgactaagaagccccggctaactacgtgccagcagccgcggtaatacgtagggggcaagcgttatccggatttactgggtgtaaagggagcgtagacggcgatgcaagccagatgtgaaagcccggggctcaaccccgggactgcatttggaactgcgtggctggagtgtcggagaggcaggcggaattcctagtgtagcggtgaaatgcgtagatattaggaggaacaccagtggcgaaggcggcctgctggacgatgactgacgttgaggctcgaaagcgtggggagcaaacaggattagataccctggtagtccacgccgtaaacgatgactactaggtgtcgggtggcaaggccattcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctgatcttgacatcccgatgccaaagcgcgtaacgcgctctttcttcggaacatcggtgacaggtggtgcatggttgtcgtcagctcgtgtcgtgagatgttgggttaagtcccgcaacgagcgcaacccctatcttcagtagccagcattatggatgggcactctggagagactgccagggagaacctggaggaaggtggggatgacgtcaaatcatcatgccccttatgaccagggctacacacgtgctacaatggcgtaaacaaagggaggcgaacccgcgagggtgggcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagcccggtgacccaacccgtaaggagggagccgtcgaagtgggaccgataactggggtgaa

SEQ ID NO: 2 - DNA, 1452 bp, Artificial Sequence
  Blautia producta
gctcaggatgaacgctggcggcgtgcttaacacatgcaagtcgagcgaagcactaagacggatttcttcggattgaagtctttgtgactgagcggcggacgggtgagtaacgcgtgggtaacctgcctcatacagggggataacagttagaaatgactgctaataccgcataagcgcacaggaccgcatggtctggtgtgaaaaactccggtggtatgagatggacccgcgtctgattagctagttggaggggtaacggcccaccaaggcgacgatcagtagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggaagagcaagtctgatgtgaaaggctggggcttaaccccaggactgcattggaaactgttgttctagagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcgggtggcaaagccattcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatccctctgaccgtcccgtaacgggggcttcccttcggggcagaggagacaggtggtgcatggttgtcgtcagctcgtgtcgtgagatgttgggttaagtcccgcaacgagcgcaacccttatccttagtagccagcacatgatggtgggcactctagggagactgccggggataacccggaggaaggcggggacgacgtcaaatcatcatgccccttatgatttgggctacacacgtgctacaatggcgtaaacaaagggaagcgagacagcgatgttgagcgaatcccaaaaataacgtcccagttcggactgcagtctgcaactcgactgcacgaagctggaatcgctagtaatcgcggatcagaatgccgcggtgaatacgttcccgggtcttgtacacaccgcccgtcacaccatgggagtcagtaacgcccgaagtcagtgacctaaccgaaaggaaggagctgccgaagcgggaccgataac

SEQ ID NO: 3 - DNA, 1456 bp, Artificial Sequence
  Blautia producta
caggatgaacgctggcggcgtgcttaacacatgcaagtcgagcgaagcgctaagacggatttcttcggattgaagtctttgtgactgagcggcggacgggtgagtaacgcgtgggtaacctgcctcatacagggggataacagttagaaatgactgctaataccgcataagcgcacaggatcgcatggtctggtgtgaaaaactccggtggtatgagatggacccgcgtctgattagctagttggaggggtaacggcccaccaaggcgacgatcagtagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggaagagcaagtctgatgtgaaaggctggggcttaaccccaggactgcattggaaactgttgttctagagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcgggtggcaaagccattcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatccctctgaccgtcccgtaacgggggcttcccttcggggcagaggagacaggtggtgcatggttgtcgtcagctcgtgtcgtgagatgttgggttaagtcccgcaacgagcgcaacccttatccttagtagccagcacatgatggtgggcactctagggagactgccggggataacccggaggaaggcggggacgacgtcaaatcatcatgccccttatgatttgggctacacacgtgctacaatggcgtaaacaaagggaagcgagacagcgatgttgagcgaatcccaaaaataacgtcccagttcggactgcagtctgcaactcgactgcacgaagctggaatcgctagtaatcgcggatcagaatgccgcggtgaatacgttcccgggtcttgtacacaccgcccgtcacaccatgggagtcagtaacgcccgaagtcagtgacctaaccgaaaggaatgagctgccgaagcgggaccgataactggggtg

SEQ ID NO: 4 - DNA, 1456 bp, Artificial Sequence
  Blautia producta
ggctcaggatgaacgctggcggcgtgcttaacacatgcaagtcgagcgaagcgctaagacggatttcttcggattgaagtctttgtgactgagcggcggacgggtgagtaacgcgtgggtaacctgcctcatacagggggataacagttagaaatgactgctaataccgcataagcgcacaggaccgcatggtctggtgtgaaaaactccggtggtatgagatggacccgcgtctgattagctagttggaggggtaacggcccaccaaggcgacgatcagtagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggaagagcaagtctgatgtgaaaggctggggcttaaccccaggactgcattggaaactgttgttctagagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcgggtggcaaagccattcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatccctctgaccgtcccgtaacggggacttcccttcggggcagaggagacaggtggtgcatggttgtcgtcagctcgtgtcgtgagatgttgggttaagtcccgcaacgagcgcaacccttatccttagtagccagcacatgatggtgggcactctagggagactgccggggataacccggaggaaggcggggacgacgtcaaatcatcatgccccttatgatttgggctacacacgtgctacaatggcgtaaacaaagggaagcgagacagcgatgttgagcgaatcccaaaaataacgtccagttcggactgcagtctgcaactcgactgcacgaagctggaatcgctagtaatcgcggatcagaatgccgcggtgaatacgttcccgggtcttgtacacaccgcccgtcacaccatgggagtcagtaacgcccgaagtcagtgacctaaccgaaaggaaggagctgccgaagcgggaccgataactggg

SEQ ID NO: 5 - DNA, 1401 bp, Artificial Sequence
  Blautia producta
gacggatttcttcggattgaagtctttgtgactgagcggcggacgggtgagtaacgcgtgggtaacctgcctcatacagggggataacagttagaaatgactgctaataccgcataagcgcacaggaccgcatggtctggtgtgaaaaactccggtggtatgagatggacccgcgtctgattagctagttggaggggtaacggcccaccaaggcgacgatcagtagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggaagagcaagtctgatgtgaaaggctggggcttaaccccaggactgcattggaaactgttgttctagagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcgggtggcaaagccattcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatccctctgaccgtcccgtaacgggggcttcccttcggggcagaggagacaggtggtgcatggttgtcgtcagctcgtgtcgtgagatgttgggttaagtcccgcaacgagcgcaacccttatccttagtagccagcacatgatggtgggcactctagggagactgccggggataacccggaggaaggcggggacgacgtcaaatcatcatgccccttatgatttgggctacacacgtgctacaatggcgtaaacaaagggaagcgagacagcgatgttgagcgaatcccaaaaataacgtcccagttcggactgcagtctgcaactcgactgcacgaagctggaatcgctagtaatcgcggatcagaatgccgcggtgaatacgttcccgggtcttgtacacaccgcccgtcacaccatgggagtcagtaacgcccgaagtcagtgacctaaccgaaaggaaggagctgccgaaagcgggaccgataactggg

SEQ ID NO: 6 - DNA, 1500 bp, Artificial Sequence
  Enterococcus faecium
ggctcaggacgaacgctggcggcgtgcctaatacatgcaagtcgtacgcttctttttccaccggagcttgctccaccggaaaaagaagagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcattcagttgggcactctagcaagactgccggtgacaaaccggaggaaggtggggatgacgtcaaatcatcatgccccttatgacctgggctacacacgtgctacaatgggaagtacaacgagttgcgaagtcgcgaggctaagctaatctcttaaagcttctctcagttcggattgcaggctgcaactcgcctgcatgaagccggaatcgctagtaatcgcggatcagcacgccgcggtgaatacgttcccgggccttgtacacaccgcccgtcacaccacgagagtttgtaacacccgaagtcggtgaggtaacctttttggagccagccgcctaagtgggatagatgattggggtgaagtcgtaaca

SEQ ID NO: 7 - DNA, 1444 bp, Artificial Sequence
  Blautia faecis
ggctcaggatgaacgctggcggcgtgcttaacacatgcaagtcgaacgggaaacattttattgaagcttcggcggatttaatttgtttctagtggcggacgggtgagtaacgcgtgggtaacctgccttatactgggggataacagccagaaatgactgctaataccgcataagcgcacagaaccgcatggttcggtgtgaaaaactccggtggtataagatggacccgcgttggattagctagttggcagggcagcggcctaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagataatgacggtacctgactaagaagccccggctaactacgtgccagcagccgcggtaatacgtagggggcaagcgttatccggatttactgggtgtaaagggagcgtagacggcgcagcaagtctgatgtgaaaggcaggggcttaacccctggactgcattggaaactgctgtgcttgagtgccggaggggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcagggagcacagctctttggtgccgccgcaaacgcattaagtattccacctggggagtacgttcgcaagaatgaaactcaaaggaattgacggggacccgcacaagcggtggagcatgtggtttaattcgaagcaacgcgaagaaccttaccaaatcttgacatccctctgaccgggacttaaccgtccctttccttcgggacaggggagacaggtggtgcatggttgtcgtcagctcgtgtcgtgagatgttgggttaagtcccgcaacgagcgcaacccctatccttagtagccagcacgcaatggtgggcactctgaggagactgccagggataacctggaggaaggcggggatgacgtcaaatcatcatgccccttatgatttgggctacacacgtgctacaatggcgtaaacaaagggaagcgaacccgcgagggtgggcaaatctcaaaaataacgtcccagttcggactgcagtctgcaactcgactgcacgaagctggaatcgctagtaatcgcggatcagaatgccgcggtgaatacgttcccgggtcttgtacacaccgcccgtcacaccatgggagtcagtaacgcccgaagtcagtgacctaactgcaaggaggagctgccgaagcgggac

SEQ ID NO: 8 - DNA, 1443 bp, Artificial Sequence
  Proteus terrae
taacacatgcaagtcgagcggtaacaggagaaagcttgctttcttgctgacgagcggcggacgggtgagtaatgtatggggatctgcccgatagagggggataactactggaaacggtggctaataccgcatgacgtctacggaccaaagcaggggctcttcggaccttgcgctatcggatgaacccatatgggattagctagtaggtgaggtaatggctcacctaggcgacgatctctagctggtctgagaggatgatcagccacactgggactgagacacggcccagactcctacgggaggcagcagtggggaatattgcacaatgggcgcaagcctgatgcagccatgccgcgtgtatgaagaaggccttagggttgtaaagtactttcagcggggaggaaggtgttaagattaatactcttagcaattgacgttacccgcagaagaagcaccggctaactccgtgccagcagccgcggtaatacggagggtgcaagcgttaatcggaattactgggcgtaaagcgcacgcaggcggtcaattaagtcagatgtgaaagccccgagcttaacttgggaattgcatctgaaactggttggctagagtcttgtagaggggggtagaattccacgtgtagcggtgaaatgcgtagagatgtggaggaataccggtggcgaaggcggccccctggacaaagactgacgctcaggtgcgaaagcgtggggagcaaacaggattagataccctggtagtccacgctgtaaacgatgtcgatttagaggttgtggtcttgaaccgtggcttctggagctaacgcgttaaatcgaccgcctggggagtacggccgcaaggttaaaactcaaatgaattgacgggggcccgcacaagcggtggagcatgtggtttaattcgatgcaacgcgaagaaccttacctactcttgacatccagagaatcctttagagatagaggagtgccttcgggaactctgagacaggtgctgcatggctgtcgtcagctcgtgttgtgaaatgttgggttaagtcccgcaacgagcgcaacccttatcctttgttgccagcgcgtgatggcgggaactcaaaggagactgccggtgataaaccggaggaaggtggggatgacgtcaagtcatcatggcccttacgagtagggctacacacgtgctacaatggcagatacaaagagaagcgacctcgcgagagcaagcggaactcataaagtctgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtagtagcttaaccttcgggagggcgcttaccactttgtgattcatgactggggtg

SEQ ID NO: 9 - DNA, 26 bp, Artificial Sequence
  Clostridium Difficile forward primer
cagtcggattgcaagtaattgacaat

SEQ ID NO: 10 - DNA, 29 bp, Artificial Sequence
  Clostridium Difficile reverse primer
agtagtatctactaccattaacagtctgc

SEQ ID NO: 11 - DNA, 19 bp, Artificial Sequence
  Clostridium scindens forward primer
ctacggctacgggcagatg

SEQ ID NO: 12 - DNA, 20 bp, Artificial Sequence
  Clostridium scindens reverse primer
ggatgtaccactggccttct

SEQ ID NO: 13 - DNA, 20 bp, Artificial Sequence
  Blautia producta forward primer
cccggggagtaaagttgaat

SEQ ID NO: 14 - DNA, 20 bp, Artificial Sequence
  Blautia producta reverse primer
cgtcaggaaaagtggcttct

SEQ ID NO: 15 - DNA, 20 bp, Artificial Sequence
  Blautia faecis forward primer
tgagacgaaatccagtgcag

SEQ ID NO: 16 - DNA, 26 bp, Artificial Sequence
  Blautia faecis reverse primer
cagtcggattgcaagtaattgacaat